(19) United States
(12) Patent Application Publication
(10) Pub. No.: US 2025/0276088 A1
(43) Pub. Date: Sep. 4, 2025

(54) SYNERGISTIC COMBINATION OF RDCFV2 AND RDCVF2L FOR THE TREATMENT OF TAUOPATHIES

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); SORBONNE UNIVERSITÉ, Paris (FR)

(72) Inventors: Thierry LEVEILLARD, Paris (FR); Céline NOUVEL, Paris (FR); Emmanuelle CLERIN-LACHAPELLE, Paris (FR); Farah OUECHTATI, Paris (FR); Géraldine MILLET-PUEL, Paris (FR)

(21) Appl. No.: 18/569,655

(22) PCT Filed: Jul. 6, 2022
(86) PCT No.: PCT/EP2022/068757

§ 371 Date: Dec. 13, 2023

(30) Foreign Application Priority Data
Jul. 7, 2021 (EP) 21184274.5

(51) Int. Cl. A61K 48/00; A61K 38/17; A61P 25/28; C12N 15/86

(52) U.S. Cl. CPC A61K 48/005; A61K 38/1709; A61P 25/28; C12N 15/86; C12N 2750/14143

(57) ABSTRACT

The two nucleoredoxin genes, NXNL1 and NXNL2 encode by alternative splicing for a secreted truncated thioredoxin that mediates neuronal survival and a thioredoxin enzyme that regulates the phosphorylation of TAU. Behavioral analyses of young Nxnl2^{-/-} mice demonstrate that this gene is involved in regulating of brain functions and is essential for learning and memory exerting positive effects on long-term potentiation (LTP) in the hippocampus. LTP dysfunction on the young Nxnl2^{-/-} mice can be fully corrected by the synergistic action of the two products of the Nxnl2 gene. Aging Nxnl2^{-/-} mice have brain stigmata of tauopathy as seen by oligomerization, phosphorylation and aggregation of TAU. This late occurring tauopathy can be prevented, by recombinant AAVs encoding RdCVF2 and RdCVF2L when administrated to young animals, which is of significant interest for therapeutic perspectives. Therefore, the present invention relates to a method of treating a tauopathy in a patient in need thereof comprising administering to said patient a therapeutically effective amount of a first polynucleotide encoding for the short isoform of the NXNL2 gene. Rod-derived Cone Viability Factor (RdCVF2) and of a second polynucleotide encoding the long isoform of the NXNL2 gene, RdCVF2L.

### SEQUENCE LISTING

This application includes as the Sequence Listing the complete contents of the XML file “SequencesXML_21286PCT”, created Jul. 10, 2024, containing 3,067 bytes, hereby incorporated by reference.

### FIELD OF THE INVENTION

The present invention is in the field of medicine, in particular neurology.

### BACKGROUND OF THE INVENTION

The nucleoredoxin-like 2 (NXNL2) gene, the paralogue of NXNL1, expresses two protein products by alternative splicing (Chalmel et al., 2007), the short rod-derived cone viability factor 2 (RdCVF2) and the longer thioredoxin-related protein RdCVF2L. We have previously reported that the inactivation of the Nxnl2 gene in the mouse results in a progressive deficit of vision and olfaction (Jaillard et al., 2012). The extracellular protein RdCVF2 improves cone survival in vitro and the RdCVF2L protein prevents in vivo the phosphorylation of the microtubule associated protein τ (TAU), induced in the retina in response to light damage (Elachouri et al., 2015). TAU is an intrinsically disordered protein which facilitates the assembly and stability of neuronal microtubules. Under pathological conditions, collectively known as tauopathies, TAU becomes hyperphosphorylated and detaches from microtubules, leading to the misfolding and formation of TAU aggregates forming neurofibrillary tangles (NFT). These are the hallmarks of several neurodegenerative diseases, such as Alzheimer's disease (AD) and frontotemporal dementia with parkinsonism linked to chromosome 17 (FTDP-17).

In the retina, considered an extension of the brain, the role of NXNL2 is partially redundant to that of NXNL1. NXNL1 which encodes two products through alternative splicing: RdCVF a protein secreted by rods photoreceptors and RdCVFL protecting rods and cones against damaging oxidation (Cronin et al., 2010; Mei et al., 2016). The delivery of both products of the NXNL1 gene, RdCVF and RdCVFL, is a promising future therapy for treating a broad range of retinal diseases, independent of genetic mutations (Clerin et al., 2020). The protective effect of RdCVF on cones results from its ability to stimulate cones' glucose uptake via its interaction at the cell-surface of the cell with a complex formed between basigin-1 (BSG1) and the glucose transporter GLUT1 (SLC2A1) (Ait-Ali et al., 2015). Glucose taken up by cones is metabolized through aerobic glycolysis, a partial anabolic metabolic pathway required for the renewal of the outer segments of photoreceptors, the neuronal structure where reside the light sensing opsins (Chinchore et al., 2017; Leveillard, 2015). RdCVFL interacts physically with TAU in the retina and prevents its phosphorylation and aggregation (Cronin et al., 2010; Fridlich et al., 2009). The presumed thiol-oxidoreductase activity of RdCVFL relies on the production of NADPH by the metabolism of glucose through the pentose phosphate pathway (PPP) (Miller et al., 2018), so the action of RdCVF via BSG1/GLUT1 potentiates the redox power of the thioredoxin- related protein RdCVFL (Leveillard and Ait-Ali, 2017). The two intricate activities of the NXNL1 gene products in the retina are essential to protect photoreceptors against starvation and oxidative damages constituting an endogenous neuroprotective metabolic and redox signaling (Leveillard and Sahel, 2017).

### SUMMARY OF THE INVENTION

The present invention is defined by the claims. In particular, the present invention relates to a method of treating a tauopathy in a patient in need thereof comprising administering to said patient a therapeutically effective amount of a first polynucleotide encoding for the short isoform of the NXNL2 gene, Rod-derived Cone Viability Factor (RdCVF2) and of a second polynucleotide encoding the long isoform of the NXNL2 gene, RdCVF2L.

### DETAILED DESCRIPTION OF THE INVENTION

The two nucleoredoxin genes, NXNL1 and NXNL2 encode by alternative splicing for a secreted truncated thioredoxin that mediates neuronal survival and a thioredoxin enzyme that regulates the phosphorylation of TAU. Behavioral analyses of young Nxnl2^{-/-} mice demonstrate that this gene is involved in regulating of brain functions and is essential for learning and memory exerting positive effects on long-term potentiation (LTP) in the hippocampus. LTP dysfunction on the young Nxnl2^{-/-} mice can be fully corrected by the synergistic action of the two products of the Nxnl2 gene. The expression pattern of the Nxnl2 gene in the brain, studied by using a Nxnl2 reporter mouse line shows a predominant expression in circumventricular organs, such as the area postrema. This fenestrated organ occupies a central position at the interface of blood circulation and the flow of cerebrospinal fluid. Glucose metabolism of the hippocampus of young Nxnl2^{-/-} mice is abnormal, as shown by metabolomic analyses of hippocampal tissue specimens. Aging Nxnl2^{-/-} mice have brain stigmata of tauopathy as seen by oligomerization, phosphorylation and aggregation of TAU. This late occurring tauopathy can be prevented, although at modest efficacy, by recombinant AAVs encoding RdCVF2 and RdCVF2L when administrated to young animals, which is of significant interest for therapeutic perspectives.

### MAIN DEFINITIONS

As used herein, the term “patient” or “patient in need thereof”, is intended for a human or non-human mammal. Typically, the patient is affected or likely to be affected with a tauopathy.

As used herein, the term “tauopathy” has its general meaning in the art. It refers to the class of neurodegenerative diseases associated with the pathological aggregation of tau protein in the brain. Tauopathies include, but are not limited to, Alzheimer's disease, traumatic brain injury, frontotemporal dementia, including the subtype of frontotemporal dementia and Parkinsonism linked to chromosome 17 (FTDP-17), progressive supranuclear palsy, corticobasal degeneration, Pick's disease, and agyrophilic grain disease. In a particular embodiment, said tauopathy is selected from the group consisting of Alzheimer's disease and traumatic brain injury.

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double-and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “RdCVF2” has its general meaning in the art and refers to the rod- derived cone viability factor 2 or Nucleoredoxin-like protein 2. An exemplary amino acid sequence for RdCVF2 is shown as SEQ ID NO:1.

As used herein, the term “RdCVF2L” has its general meaning in the art and refers to the rod- derived cone viability factor 2 long isoform. An exemplary amino acid sequence for RdCVF2L is shown as SEQ ID NO:2.

>NXNL2_HUMAN Nucleoredoxin-like protein 2
OS = Homo sapiens
SEQ ID NO: 1
MVDILGERHLVTCKGATVEAEAALQNKVVALYFAAARCAPSRDFT
PLLCDFYTALVAEARRPAPFEVVFVSADGSSQEMLDFMRELHGAW
LALPFHDPYRH
>sp|Q5VZ03-1|NXNL2_HUMAN Nucleoredoxin-
like protein 2
OS = Homo sapiens
OX = 9606 GN = NXNL2
PE = 2 SV = 1
SEQ ID NO: 2
MVDILGERHLVTCKGATVEAEAALQNKVVALYFAAARCAPSRDFT
PLLCDFYTALVAEARRPAPFEVVFVSADGSSQEMLDFMRELHGAW
LALPFHDPYRHELRKRYNVTAIPKLVIVKQNGEVITNKGRKQIRE
RGLACFQDWVEAADIFQNFSV

As used herein, the term “vector” refers to an agent capable of delivering and expressing the transgene in a host cell. The vector may be extrachromosomal (e.g. episome) or integrating (for being incorporated into the host chromosomes), autonomously replicating or not, multi or low copy, double-stranded or single-stranded, naked or complexed with other molecules (e.g. vectors complexed with lipids or polymers to form particulate structures such as liposomes, lipoplexes or nanoparticles, vectors packaged in a viral capsid, and vectors immobilized onto solid phase particles, etc.). The definition of the term “vector” also encompasses vectors that have been modified to allow preferential targeting to a particular host cell. A characteristic feature of targeted vectors is the presence at their surface of a ligand capable of recognizing and binding to a cellular and surface-exposed component such as a cell-specific marker, a tissue- specific marker or a cell-specific marker.

As used herein, the term “viral vector” encompasses vector DNA as well as viral particles generated thereof. Viral vectors can be replication-competent, or can be genetically disabled so as to be replication-defective or replication-impaired. The term “replication-competent” as used herein encompasses replication-selective and conditionally-replicative viral vectors which are engineered to replicate better or selectively in specific host cells (e.g. tumoral cells).

As used herein, the term “AAV” has its general meaning in the art and refers to adeno- associated virus, and may be used to refer to the virus itself or derivatives thereof. The term covers all serotypes and variants both naturally occurring and engineered forms. The term “AAV” includes but is not limited to AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), and AAV type 8 (AAV-8).) and, AAV type 9 (AAV9). The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077 (AAV-1), AF063497 (AAV-1), NC_001401 (AAV-2), AF043303 (AAV-2), NC_001729 (AAV-3), NC_001829 (AAV-4), U89790 (AAV-4), NC_006152 (AAV-5), AF513851 (AAV-7), AF513852 (AAV-8), and NC_006261 (AAV-8).

As used herein, the term “rAAV” refers to recombinant adeno-associated virus, also referred to as a recombinant AAV vector (or “rAAV vector”). The term thus refers to an AAV vector comprising the transgene of interest for the genetic transformation of a cell. In general, the rAAV vectors contain 5′ and 3′ adeno-associated virus inverted terminal repeats (ITRs), and the transgene of interest operatively linked to sequences which regulate its expression in a target cell.

As used herein, the term “pseudotyped AAV vector” refers to a vector particle comprising a native AAV capsid including an rAAV vector genome and AAV Rep proteins, wherein Cap, Rep and the ITRs of the vector genome come from at least 2 different AAV serotypes.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” refers to the amount of the expression level of the polynucleotide sufficient to confer its therapeutic or beneficial effect(s) in the host receiving said polynucleotide. Expression levels of the polynucleotide can be measured at the protein or the mRNA level using methods known in the art. The doses of vectors may be easily adapted by the skilled artisan, e.g., depending on the tauopathy to be treated, the subject (for example, according to his weight, metabolism, etc.), the treatment schedule, etc. A preferred effective dose within the context of this invention is a dose allowing an optimal transduction of brain cells. Typically, from 10⁸ to 10¹² viral genomes (transducing units) are administered per dose in mice, preferably from about 10⁹ to 10¹¹. Typically, the doses of AAV vectors to be administered in humans may range from 10⁸ to 10¹² viral genomes, most preferably from 10⁹ to 10¹¹.

### METHODS OF THE PRESENT INVENTION

The first object of the present invention relates to a method of treating a tauopathy in a patient in need thereof comprising administering to the patient a therapeutically effective amount of a first polynucleotide encoding for the short isoform of the NXNL2 gene, Rod-derived Cone Viability Factor (RdCVF2) and of a second polynucleotide encoding for the long isoform of the NXNL2 gene, RdCVF2L.

In some embodiments, the patient is at the stage of mild-cognitive impairment as assessed by any method well-known in the art.

In some embodiments, the first polynucleotide encodes for the polypeptide having the amino acid sequence as set forth in SEQ ID NO:1 and of the second polynucleotide encodes for the polypeptide having the amino acid sequence as set forth in SEQ ID NO:2.

In some embodiments, the first polynucleotide encoding for RdCVF2 and of the second polynucleotide encoding for RdCVF2L are administered by separate vectors, which can be administered simultaneously or sequentially. Therefore, the present invention also relates to a method of treating a tauopathy in a patient in need thereof comprising administering to the patient a therapeutically effective amount of a first polynucleotide encoding for RdCVF2 and of a second polynucleotide encoding for RdCVF2L wherein said first polynucleotide and second polynucleotide are contained in separate expression vectors.

In some embodiments, the first polynucleotide encoding for RdCVF2 and of the second polynucleotide encoding for RdCVF2L are administered by a single vector. Therefore, the present invention also relates to a method of treating a tauopathy in a patient in need thereof comprising administering to the patient a therapeutically effective amount of a first polynucleotide encoding for RdCVF2 and of a second polynucleotide encoding for RdCVF2L wherein said first polynucleotide and second polynucleotide are contained in a single vector.

In some embodiments, the vector of the present invention is selected from the group consisting of viral and non-viral vectors.

Typically, viral vectors include, but are not limited to polynucleotide sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus and AAV vectors. Preferred viral gene delivery vector are rAAV vectors.

In some embodiments, the AAV vector is the AAV2-7m8 as described in WO2012145601 and Dalkara D, Byrne LC, Klimczak RR, Visel M, Yin L, Merigan WH, Flannery JG, Schaffer DV. In vivo-directed evolution of a new adeno-associated virus for therapeutic outer retinal gene delivery from the vitreous. Sci Transl Med. 2013 Jun. 12;5(189):189ra76.

In some embodiments, the viral vector is a pseudotyped AAV vector. Examples of AAV chimeric vectors include but are not limited to AAV2/5, AAV2/6, and AAV2/8. In some embodiments, the AAV chimeric vector is the AAV2/8 described in U.S. Pat. No. 7,282,199, which is incorporated by reference herein.

In some embodiments, the vector may also comprise regulatory sequences allowing expression and, secretion of the encoded protein, such as e.g., a promoter, enhancer, polyadenylation signal, internal ribosome entry sites (IRES), sequences encoding protein transduction domains (PTD), and the like. In this regard, the vector comprises a promoter region, operably linked to the transgene of interest, to cause or improve expression of the protein in infected cells. Such a promoter may be ubiquitous, tissue-specific, strong, weak, regulated, chimeric, inducible, etc., to allow efficient and suitable production of the protein in the infected tissue. The promoter may be homologous to the encoded protein, or heterologous, including cellular, viral, fungal, plant or synthetic promotersExamples of such regulated promoters include, without limitation, Tet on/off element-containing promoters, rapamycin-inducible promoters and metallothionein promoters. Examples of ubiquitous promoters include viral promoters, particularly the CMV promoter, the RSV promoter, the SV40 promoter, etc. and cellular promoters such as the PGK (phosphoglycerate kinase) promoter. The promoters may also be neurospecific promoters such as the Synapsin or the NSE (Neuron Specific Enolase) promoters (or NRSE (Neuron restrictive silencer element) sequences placed upstream from the ubiquitous PGK promoter). The vector may also comprise target sequences for miRNAs achieving suppression of transgene expression in non-desired cells. For example, suppression of expression in the hematopoietic lineages (“de-targeting”) enables stable gene transfer in the transduced cells by reducing the incidence and the extent of the transgene-specific immune response (Brown BD, Nature Medicine 2008). In some embodiments, the vector comprises a leader sequence allowing secretion of the encoded protein. Fusion of the transgene of interest with a sequence encoding a secretion signal peptide (usually located at the N-terminal end of secreted polypeptides) will allow the production of the therapeutic protein in a form that can be secreted from the transduced cells. Examples of such signal peptides include the albumin, the β-glucuronidase, the alkaline protease or the fibronectin secretory signal peptides.

In some embodiments, the vector of the present invention is administered to the patient intravenously, intracerebroventricularly, intramuscularly, or intrathecally.

The vector of the present invention is administered into suitably formulated pharmaceutical composition comprising the vector and a pharmaceutically acceptable carrier, diluent, excipient, or buffer. In some embodiments, the pharmaceutically acceptable carrier, diluent, excipient, or buffer is suitable for use in a human. Such excipients, carriers, diluents, and buffers include any pharmaceutical agent that can be administered without undue toxicity. Carriers might include cationic lipids, non-ionic lipids and polyethylene glycol (PEG) as synthetic vectors to enhance siRNA delivery. siRNA might be contained in the hydrophilic interior of the particle or polyethyleneimine and derivatives can be used to fabricate both linear and branched polymeric delivery agents. Cationic polymers with a linear or branched structure can serve as efficient transfection agents because of their ability to bind and condense nucleic acids into stabilized nanoparticles. Such materials have also been shown to stimulate nonspecific endocytosis as well as endosomal escape necessary to enhance nucleic acid uptake. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H.C. Ansel et al., eds., 7th ed., Lippincott, Williams, & Wilkins; and Handbook of Pharmaceutical Excipients (2000) A.H. Kibbe et al., eds., 3 rd ed. Amer. Pharmaceutical Assoc. The invention will be further illustrated by the following figures and examples. However, these examples and figures should not be interpreted in any way as limiting the scope of the present invention.

### FIGURES

FIG. 1: Treatment of the Nxnl2^{-/-} mouse with recombinant AAVs encoding RdCVF2 and RdCVF2L. A. Recording of fEPSP normalized to the baseline for AAV2/9-2YF-CMV/CBA-GFP, injected to ♂ Nxnl2^{+/+} and Nxnl2^{-/-} mice at 2-month and of ♂ Nxnl2^{-/-} mice at 2-month, injected with AAV2/9-2YF-CMV/CBA-RdCVF22AGFP. B. Comparison of the normalized fEPSP recording of ♂ Nxnl2^{-/-} and Nxnl2^{+/+} mice injected with AAV2/9-2YF-CMV/CBA-GFP with ♂ Nxnl2^{-/-} injected with AAV2/9-2YF-CMV/CBA-RdCVF2L^{2A}GFP. C. Comparison of the normalized fEPSP recording of ♂ Nxnl2^{-/-} and Nxnl2^{+/+} mice injected with AAV2/9-2YF-CMV/CBA-GFP, with ♂ Nxnl2^{-/-} injected with half the dose used previously (½) of AAV2/9-2YF-CMV/CBA-RdCVF2^{2A}GFP combined with ½ of AAV2/9-2YF-CMV/CBA-RdCVF2L^{2A}GFP. The data are plotted with SEM. The LTP data were analyzed using two-way ANOVA—repeated measures. The metabolomic data were analyzed using one-way ANOVA.

FIG. 2: Phosphorylation of TAU using AT100 antibody in the brain of treated 18-months Nxnl2^{-/-} mice. A. Expression of GFP in whole brain extracted of treated Nxnl2^{-/-} mice at 18 months of age. The sex of the animals is indicated. B. Phosphorylation of TAU using AT100 antibody in whole brain extracts of treated Nxnl2^{-/-} mice at 18 months of age. The sex of additional Nxnl2^{-/-} mice (10 and 11 in bold) is indicated. C. Comparison of the level of phosphorylation of TAU using AT100 antibody in whole brain extracts of untreated 18-months Nxnl2^{-/-} mice to that of Nxnl2^{-/-} mice at 18 months of age treated with ½ AAV2/9-2YF-CMV/CBA-RdCVF2^{2A}GFP+½ of AAV2/9-2YF-CMV/CBA-RdCVF2L^{2A}GFP. Data were analyzed using one-way ANOVA.

FIG. 3: Hypothetical working model. Under non pathological conditions, a satiety hormone is triggering the release of rod-derived cone viability factor 2 (RdCVF2) from the area postrema to the 4th ventricle. RdCVF2 circulates in the cerebrospinal fluid and reach its cell surface receptor on hippocampal pyramidal neurons increasing glucose uptake via GLUT4. Aerobic glycolysis participates in the membrane surface increase to form new dendritic spines. Metabolism of this glucose by the pentose phosphate pathway (PPP) increases the redox power of thioredoxin, such as RdCVF2L that reduces TAU aggregation resulting from increase oxidative stress during aging.

### EXAMPLE

### Methods

### Animals

The Nxnl2^{-/-} mice on BALB/c background were generated previously (Jaillard et al., 2012). The BALB/c (Nxnl2^{+/+}) mice were used as their wild-type controls. The Nxnl2^{R/R} mice was generated at the Institut Clinique de la Souris http://www.ics-mci.fr/en/using embryonic stem cell clones on a C57BL/6@N background from the VelociGene project #VG14768 MMRRC:059676-UCD. These clones were produced using bacterial artificial chromosome (BAC)-based targeting vectors were constructed to replace the coding sequence of the Nxnl2 gene with a β-galactosidase reporter gene at positions (51,266,695-51,270,168) of the mouse chromosome 13, corresponding to the ATG and TGA of the RdCVF2L mRNA (Valenzuela et al., 2003). The mice, generated on a C57BL/6@N background, were genotyped using multiplex PCR. The heterozygous mice (Nxnl2^{R/+}) were produced by crossing with C57BL/6@N, wild-type mice, which were using as negative controls (Nxnl2^{+/+}, C57BL/6N). The mouse lines were maintained at the animal facility Charles Foix (UMS28) under standard conditions with access ad libitum to food and water with a 12-h light/dark cycle. The animals under experimentation were transferred to the animal facility of the Institut de la Vision under the agreement obtained April 26th 2016 and for 5 years of the direction départementale de la protection des populations de Paris (B-75-12-02) and principal investigator (T.L.) certificate (N^{o}A-75-1863; OGM n^{o}5080 CA-II). Mice were housed with access ad libitum to food and water with a 12-h light/dark cycle of 20-50 1x. The use of genetically modified organisms was declared and authorized by the Ministère de l'Enseignement supérieur, de la Recherche et de l'Innovation (#4503 bis December 20^{th} 2018). The projects were reviewed by the ethic committee Charles Darwin N^{o}5 and authorized by the Ministère de l'Enseignement supérieur, de la Recherche et de l'Innovation (APAFIS #748 and APAFIS #749). The intracardiac injections for fEPSP recording were done at the at Centre d'Exploration et de Recherche Fonctionnelle Expérimentale (CERFE) under the agreement APAFIS #4837. fEPSP recording was performed at the Institut de Pharmacologie Moléculaire et Cellulaire under the agreement APAFIS #15028. Behavior tests were performed at the Institut Clinique de la Souris. All experiments were performed in accordance with the European Community Council Directives of Sep. 22, 2010 (2010/63/UE).

### Behavior Testing

The tests were performed on groups of 12 ♂ Nxnl2^{-/-} and Nxnl2^{+/+} aged of 2 months at the department of Phenotyping of the Institut Clinique de la Souris (Phenotyping—Institut Clinique de la Souris (ics-mci.fr) using standardized procedures and a well-established pipeline (Dubos et al., 2018; Duchon et al., 2021; Lamprianou et al., 2006; Zhang et al., 2020). The tests were performed following an ordered process: 1—Spontaneous activity and food/water intake, 2—Open-field test (Anxiety-related and social behavior), 3—SHIRPA (General health and basic sensory functions), 4—Grip test (Sensori-motor abilities), 5—Traction reflex test/String test (Sensori-motor abilities), 6—Rotarod test (Sensori-motor abilities), 7—Y-maze spontaneous alternation (Learning and memory), 8—Tail suspension test (Depression-like behavior), 9—Acoustic startle reactivity and pre-pulse inhibition, 10—Contextual and cued fear conditioning (Learning and memory), 11—Hot plate test (Pain sensitivity), 12—Pentylenetetrazol susceptibility. The water Morris maze test (Learning and memory) was performed on a distinct cohort of 12 ♂ Nxnl2^{-/-} and Nxnl2⁺⁺ aged of 2 months. The Y-maze spontaneous alternation was also performed on an additional cohort of 12 ♂ Nxnl2^{-/-} and Nxnl2^{+/+} aged of 2 months and on a cohort of 12 ♂ Nxnl1^{-/-} and Nxnl1^{+/+} aged of 2 months. On testing days, animals were transferred to the antechambers of the experimental room 30 min before the start of the experiment. All experiments were performed between 8:00 AM and 4:00 PM. A resting period of 2 days to 1 week was used between two consecutive tests. Row data are available at https://data.mendeley.com/datasets/y6d6zsgfyv/1.

### LTP Recording

Recordings were performed on hippocampal slices of groups of ♂ Nxnl2^{-/-} and Nxnl2^{+/+} aged of 2 months at E-Phy-Science https://www.e-phy-science.com/. After delivery, Mice were housed in standard ventilated cages (IVC, Sealsafe, Techniplast) coupled to an air-handling unit (TouchSLIMline, Exhaust, Techniplast), equipped with solid floors and a layer of bedding. The cages were cleaned at regular intervals to maintain hygiene. Environmental parameters were as follows: temperature: ˜22° C., relative humidity: ˜55%. Mice had ad libitum access to standard rodent chow. The food was stored under dry and cool conditions in a well-ventilated storage room. Mice had ad libitum access to pre-filtered and sterile water. The amounts of food and water were checked daily, supplied when necessary and refreshed once a week. Mice were kept on a 12-h light/dark cycle. Mice were deeply anesthetized with isoflurane and decapitated. The brain was quickly removed and immersed in ice-cold pre-oxygenated artificial cerebrospinal fluid (aCSF). 400 μm-thick slices were prepared using a vibratome (VT 1000S; Leica Microsystems, Bannockburn, IL), and placed in a holding chamber in aCSF containing: 124 mM NaCl, 3.5 mM KCl, 1.5 mM MgSO₄, 2.5 mM CaCl₂, 26.2 mM NaHCO₃, 1.2 mM NaH₂PO₄, 11 mM glucose, continuously oxygenated (pH=7.4, 27° C.). Slices were allowed to recover in these conditions from the slicing at least 1 h before recording. For electrophysiological recordings, a single slice was placed in the recording chamber, submerged and continuously superfused with gassed (95% O₂, 5% CO₂) aCSF (28-31° C.) at a constant rate (2 ml min⁻¹) for the reminder of the experiment. Extracellular fEPSPs were recorded in the CA1 stratum radiatum using a glass micropipette filled with aCSF. fEPSPs were evoked by the electric stimulation of Schaffer collaterals/commissural pathway at 0.1 Hz with a bipolar tungsten stimulating electrode placed in the stratum radiatum (100 μs duration). Stable baseline fEPSPs were recorded by stimulating at 30% maximal field amplitude for 20 min prior to beginning experiments [single stimulation every 20 s (3 Hz)]. Synaptic transmission (input/output) curves were constructed to assess basal synaptic transmission in groups of animals. LTP was induced by the following stimulation protocol: 3 trains of 100 stimulations at 100 Hz at the same stimulus intensity, with a 20 s interval between trains. Following this conditioning stimulus, a 1 h test period was recorded where responses were again elicited by a single stimulation every 20 s (3 Hz) at the same stimulus intensity. Signals were amplified with an Axopatch 200B amplifier (Molecular Devices, Union City, CA) digitized by a Digidata 1550 interface (Axon Instruments, Molecular Devices, US) and sampled at 10 kHz. Recordings were acquired using Clampex (Molecular Devices) and analyzed with Clampfit (Molecular Devices). Experimenters were blinded to genotype and treatment for all experiments. Data were analyzed by measuring the slope of individual fEPSPs at 0-1.5 ms from the top of the signal by linear fitting using Clampfit (Molecular Devices). LTP was quantified by comparing the mean fEPSP slope over the post-HFS period with the mean fEPSP slope during the baseline period. Group effects was assessed by changes in fEPSP slope, expressed as the percentage of the baseline value. Recordings were also performed on hippocampal slices of groups of ♂ Nxnl2^{-/-} and Nxnl2^{+/+} aged of 2 months at Institut du Fer à Moulin https://ifm-institute.org/en/home/according to a protocol previously described (Le Roux et al., 2013). Row data are available at https://data.mendeley.com/datasets/2rprjfnvk4/1.

β-galactosidase Staining

For β-galactosidase enzymatic staining, two months aged mice were perfused by transcardial perfusion with 4% paraformaldehyde. Brains were removed and fixed by immersion in 4% paraformaldehyde for 2 h followed by incubation in sucrose 30% over-night (ON). Coronal sections were cut at 25 μm on a HM 450 sliding microtome (Thermo Scientific™). For detection of β-galactosidase, sections were rinsed in phosphate-buffered saline (PBS) 1x and incubated for 24 hours in staining solution (0.1 M ferricyanide, 0.1 M ferrocyanide, 1 M MgCl₂, 20 mg/ml 5-bromo-4-chloro-3-indolyl-β-D-galactopyranoside in PBS 1x 0.1% Tween®) at 37° C. in a humidified incubator. After washing in PBS, sections were mounted in

Fluoromount™ Aqueous Mounting Medium. Slides were scanned at cellular resolution with a Nanozoomer (Hamamatsu). The profile in the whole Nxnl2^{R/+} brain is available at EBRAINS—Expression profile of the nucleoredoxin-like 2 gene in the mouse brain using a beta-galactosidase knock-in reporter strain

### Metabolomic Analysis

Metabolomic analysis of standardized hippocampal specimens of ♂ Nxnl2^{-/-} and Nxnl2^{+/+} aged of 2 months or treated ♂ and ♀ Nxnl2^{-/-} and Nxnl2^{+/+} aged of 2 months were performed by the national infrastructure MetaToul https://www6.toulouse.inrae.fr/metatoul/. The brain is extracted from the cranium, making sure not to damage it, then rinsed in PBS. We removed the cerebellum, making sure not to damage the extremities of the 2 lobes and glued the brain on the support of the vibratome, posterior side up. Using a vibratome, we cut 0.5 mm slices lengthwise until reaching the hippocampus. We collected 0.5 mm slices with hippocampal tissue in PBS. Using a binocular magnifying glass, we selected slices with well visible hippocampus morphology (serrated gyrus of the hippocampus visible). We then made three standardized sections using a punch of 2 mm diameter, collected in an Eppendorf tube, and immediately frozen in liquid nitrogen while awaiting sample processing, then further stored at −80° C. On the day of extraction of the metabolites, we set-up the freeze-mill to cool. The tubes were taken out of −80° C. and left them in liquid nitrogen while waiting. We added 3 steel balls to the tubes containing the hippocampus specimens and immediately placed them in the previously cooled ball mill. We performed 5×1 min 30 frequency/sec on a liquid nitrogen-refrigerated CryoMill (Retsch). When tissues were powdered, we added 1 ml per tube of methanol/H₂O (80/20) previously cooled to −80° C. plus 120 μl of ¹³C internal standard. We proceed for 1 min successively with 10 sec of vortex+10 sec of sonicator+10 sec on ice. The specimens were centrifuged for 5 min at 4° C. 13,000 g. The supernatants were collected in a 2-ml Eppendorf tube to which we added 1 ml of cold methanol/H₂O (80/20) mixed to the pellet and performed the same 1 min vortex/sonicator/ice cycle, as before. We centrifuged the tubes for 5 min at 4° C. at 13,000 g, recovered the 2^{nd} supernatant and pooled it with the 1^{st} one. The resulting standardized hippocampal specimens were frozen by immersing in liquid nitrogen and stored at −80° C. pending metabolomic analysis. The specimens were sent on dry-ice. For the first experiment, intracellular metabolites were analyzed as described in (Bolten et al., 2007; Kiefer et al., 2007) Briefly, analysis was performed by high performance anion exchange chromatography (Dionex ICS 2000 system, Sunnyvale, USA) coupled to a triple quadrupole QTrap 4000 (AB Sciex, CA USA) mass spectrometer (Kiefer et al., 2007). This analytical technology allows the separation and analysis of numerous highly polar metabolites belonging to several chemical families in the same analytical run. All samples were analyzed in the negative mode by multiple reaction monitoring. The amounts of metabolites of glycolysis, pentose phosphate pathways, tricarboxylic acid cycle as well as nucleotides were determined. To ensure highly accurate quantification, the isotope dilution mass spectrometry (IDMS) method was used (Wu et al., 2005). For quantification the addition of full ¹³C E. coli extract which contains a majority of the target metabolites was used, the internal standard. The quantification for each metabolite was first expressed as ¹³C/¹²C ratio or as ¹²C area if the internal ¹³C standard was not available. For metabolites for which a chemical standard was available, the absolute quantification was calculated from the corresponding calibration curve. For the second and the third experiment, we used a LTQ Orbitrap Velos™/Liquid anion exchange chromatography Dionex™ ICS-5000+ Reagent-Free™ HPIC™ equipment. The analyses were carried out on an IC-MS platform of a liquid anion exchange chromatography Dionex™ ICS-5000+ Reagent-Free™ HPIC™ (Thermo Fisher Scientific™, Sunnyvale, CA, USA) system, coupled to a Thermo Scientific™ LTQ Orbitrap Velos™ mass spectrometer (Thermo Fisher Scientific, San Jose, CA, USA) equipped with a heated electrospray ionization probe. Liquid anion exchange chromatography was performed with the Thermo Scientific Dionex ICS-5000+ Reagent-Free HPIC system (Thermo Fisher Scientific) equipped with an eluent generator system (ICS-5000+EG, Dionex) for automatic base generation (KOH). Analytes were separated within 50 min, using a linear KOH gradient elution applied to an IonPac AS11 column (250×2 mm, Dionex) equipped with an AG11 guard column (50×2 mm, Dionex) at a flow rate of 0.35 ml/min. The gradient program was following: 0 min:0.5 mM, 1 min:0.5 mM, 9.5 min:4.1 mM, 14.6 min:4.1 mM, 24 min:9.65 mM, 31.1 min:90 mM and 43 min:90 mM, then 43 to 48 min vat 0.5 mM. The column and autosampler temperatures were thermostated at 25° C. and 4° C., respectively. The injected sample volume was 15 μl. Measures were performed in triplicates from separate specimens. Mass detection was carried out in a negative electrospray ionization (ESI) mode at a resolution of 60 000 (at 400 m/z) in full-scan mode, with the following source parameters: the capillary temperature was 350° C., the source heater temperature, 300° C., the sheath gas flow rate, 50 arbitrary units (a.u.), the auxiliary gas flow rate, 5 a.u., the S-Lens RF level, 60%, and the source voltage, 2.75 kV. Data acquisition was performed using Thermo Scientific Xcalibur software. Metabolites were determined by extracting the exact mass with a tolerance of 5-10 ppm. For quantification the addition of full ¹³C E. coli extract which contains a majority of the target metabolites was used, and quantified as above. Data were processed using TraceFinder 4.1 software. For the third experiment, the gradient was modified as follows equilibration with 7 mM KOH during 1.0 min; then KOH ramp from 7 to 15 mM, 1-9.5 min; constant concentration 10.5 min; ramp to 45 mM in 10 min; ramp to 70 mM in 3 min; ramp to 100 mM in 0.1 min; constant concentration 8.9 min; drop to 7 mM in 0.5 min; and equilibration at 7 mM KOH for 7.5 min. Processed data are available at DOI 10.17632/yjmhvpp7rf.1. Row data are available upon request.

### Recombinant AAV Production and Validation

Plasmids AAV2/9-2YF-CMV/CBA-RdCVF2^{2A}GFP and AAV2/9-2YF-CMV/CBA-RdCVF2L^{2A}GFP contain the GFP protein, a self-cleaving 2A peptide upstream of the cDNA of mouse RdCVF2 (Q9D531-4) and mouse RdCVF2L (Q9D531-3) respectively under the control of the CMV/CBA promoter. AAV2/9-2YF-CMV/CBA-GFP is the negative control. Recombinant AAV was purified via iodixanol gradient ultracentrifugation as described previously (Ait-Ali et al., 2015). The 40% iodixanol fraction was then buffer-exchanged against PBS supplemented with 0.001% tween-20 and concentrated using ultrafiltration on with a cutoff of 100 kDa (Amicon Ultra-15) to a final volume of 200 μl. DNase-resistant viral genomes in the concentrated stock were then tittered by qPCR relative to standards. Vector concentrations were calculated in viral genomes (vg)/ml with AAV2/9-2YF-CMV/CBA-GFP at 1.35×10¹⁴ vg/ml, AAV2/9-2YF-CMV/CBA-RdCVF2^{2A}GFP at 1.16×10¹⁴ vg/ml, and AAV2/9-2YF-CMV/CBA-RdCVF2L^{2A}GFP at 8.85×10¹³ vg/ml. The quality controls were performed by silver staining using ProteoSilve™ Silver Stain Kit (PROT-SIL1, Sigma) and the procedure recommended by the supplier. Each lane was loaded with 1×10¹⁰ vg with 100 mM DTT onto a 4-12% gel. Uranyl acetate straining was done according to (Grieger et al., 2016). Just before pipetting, microtubes were shaken to resuspend the virus particles. 5 μl of each sample was deposited on a 300-mesh nickel grids with 10 nm formvar and 1 nm carbon film (Electron Microscopy Sciences, USA) side up for 1 min at room temperature to let the virus particles adsorb on the film. Samples were quickly rinsed with one first drop (filtered with a 0.22 μm isopore) of 2.5% w/v uranyl acetate (Prolabo, France) diluted with ultrapure water, then contrasted 1 min with a second drop of the same solution in the dark. Excess solution was removed using a Whatman grade n^{o}40 ashless filter paper and grids were let dry at room temperature. Observations were performed using a LaB₆ JEM 2100 HC TEM (Jeol, Japan), at 200 kV, spot size 1, condenser aperture 1 (150 μm diameter) and objective aperture 3 (15 μm diameter). Acquisitions were made with a side mounted Veleta CCD camera driven by iTEM software version 5.2 (Olympus, Japan). Images were recorded with a 2k×2k pixels definition (binning 1×1) and a 750 ms exposure time. Quantification of the ratio of empty versus full particles was obtained from the mean of counting of 4 independent images taken at magnification 300,000. The intracardiac injection in PN4 mice was done as described previously (Ait-Ali et al., 2015). PN4 Nxnl2^{-/-} or Nxnl2^{+/+} mice (BALB/c) aged were injected directly in the heart with 20 μl of viral solution (4×10¹² vg) for AAV2/9-2YF-CMV/CBA-GFP, AAV2/9-2YF-CMV/CBA-RdCVF2^{2A}GFP and AAV2/9-2YF-CMV/CBA-RdCVF2L^{2A}GFP. ½ AAV2/9-2YF-CMV/CBA-RdCVF2^{2A}GFP+½ AAV2/9-2YF-CMV/CBA-RdCVF2L^{2A}GFP corresponds to 2×10¹² vg of each recombinant vector Alternatively, we injected only 10 μl of ½ AAV2/9-2YF-CMV/CBA-RdCVF2^{2A}GFP+½ AAV2/9-2YF-CMV/CBA-RdCVF2L^{2A}GFP which correspond to corresponds to 0.5×10¹² vg of each recombinant vector.

### Immunohistochemistry and Histological Staining

Two months aged Nxnl2^{R/+} and Nxnl2^{+/+} ♂ mice were perfused by transcardial perfusion with 4% paraformaldehyde. Brains were removed and fixed by immersion in 4% paraformaldehyde for 2 h followed by incubation in sucrose 30% ON. Coronal sections were cut at 25 μm on a HM 450 sliding microtome (Thermo Scientific™). After β-galactosidase staining, Nxnl2^{R/+} slices were permeabilized in 0.3% Triton X-100 in PBS for 4 min and block in 5% bovine serum albumin (BSA), 10% normal goat serum (NGS) in PBS for 1H30 at room temperature (RT). After two washes of 5 min in PBS, slides were incubated ON at 4° C. rat monoclonal anti-PLVAP (BD Biosciences Cat #553849, RRID: AB_395086, 1/20), plus rabbit polyclonal anti-GLUT1 antibodies (Alpha-diagnostic GT11-A, RRID: AB_1616630, 1/100) in 5% BSA, 10% NGS in PBS. Slides were washed twice for 10 min in 5% BSA in PBS and once 10 min in 1% BSA, 0.1% tween-20 in PBS at RT, then 1H30 at RT with anti-rat HRP (Jackson ImmunoResearch RRID: AB_2340639, 1/200) in 2% NGS 0,1% Triton in PBS or in the secondary incubation with anti-rat fluo-λ488 ( 1/400) and anti-mouse fluo-λ594 ( 1/400) in 1% BSA, 10% NGS, 0.1% tween-20. Nxnl2^{+/+} brain was dissected after intra-cardiac perfusion of in 4% paraformaldehyde/PBS (PFA 4%/PBS) with a peristaltic pump followed by the incubation of (PFA 4%/PBS) ON at 4° C. Tissues were incubated successively in 10, 20 and 30% sucrose at 4° C. and embedded in optimal cutting temperature (OCT medium) and then freezing in isopentane cooled in liquid nitrogen between −40 and 45° C. The staining protocol with hematoxylin-eosin is standard with 12 min for hematoxylin and 2 min for eosin on 12 μm horizontal cryostat sections. The brain specimens of Nxnl2^{+/+} and Nxnl2^{-/-}, were obtained after intra-cardiac perfusions as above and incubated successively in 10, 20 and 30% sucrose at 4° C. and cutting with a slide microtome (HM450, Microm) and a freezer unit with 60 μm sagittal slide. The floating sections with observation of GFP were selected for immunohistochemistry with a chicken polyclonal antibodies anti-GFP (Abcam Cat #ab13970, RRID: AB_300798, 1/1,250) ON at 4° C., then, after 2 h with saturation step with triton 0.1%/PBS and revealed with a secondary goat anti-chicken λ488 ( 1/600) and with Hoechst 33342 (Invitrogen) during 1 h at room temperature and imaged with an epifluorescence microscope (Leica). Slices were permeabilized in 0.3% Triton X-100 in PBS for 4 min and block in 5% bovine BSA, 10% NGS in PBS for 1H30 at room temperature (RT). After two washes of 5 min in PBS, slides were incubated ON at 4° C. with mouse monoclonal antibody anti-RBFOX3/NeuN (Millipore Cat #MAB377, RRID: AB_2298772, 1/500 dilution) and rabbit polyclonal anti-GFAP antibodies (Agilent Cat # N1506, RRID: AB_10013482, 1/500). Slices were washed twice in PBS then incubated 1H30 at RT with and fluo-λ488 conjugated goat anti-mouse and fluo-λ594 conjugated goat anti-rabbit antibodies.

### Western Blotting

For western blotting analysis of hippocampal specimens, Nxnl2^{-/-} and Nxnl2^{-/-} standardized hippocampal specimens or standardized hippocampal specimens from AAV-treated Nxnl2^{-/-} mice were prepared from three 0.5 mm thick vibratome slice of 2 mm ∅, all from 2-month mice. Tissues were sonicated twice 10 s on ice in 50 mM Tris-HCl (pH 7.5), 1 mM EDTA, 1 mM dithiothreitol (DTT), 1% Triton X-100, 1 mM phenylmethylsulfonyl fluoride (PMSF), 0.14 mM Tosyl-L-lysine chloromethyl ketone hydrochloride (TLCK) in the presence of a cocktail of proteinase inhibitors (P2714, Sigma). After 30 min on ice, the extracts were centrifuged 5 min at 12,000 rpm at 4° C. and 40 μg of whole cell extracted were loaded on a SDS gel and transferred to a 0.22 μm nitrocellulose membrane. The samples were not heated for GLUT1 analysis (Ait-Ali et al., 2015; Rath et al., 2009). After Saturation, the membranes were incubated with rabbit polyclonal anti-GLUT1 antibodies (Alpha-diagnostic GT11-A, RRID: AB_1616630, 1/500), rabbit monoclonal anti-GLUT3 antibody (Abcam Cat #ab191071, RRID: AB_2736916, 1/1,000), mouse monoclonal anti-GLUT4 antibody (Santa Cruz Biotechnology Cat #sc-53566, RRID: AB_629533, 1/100) or chicken polyclonal antibodies anti-GFP (Abcam Cat #ab13970, RRID: AB_300798, 1/5,000) ON at 4° C. The western blots were revealed with anti-rabbit or anti-mouse IgG coupled to peroxidase. The signals do not appear with the secondary antibody alone. After stripping the membrane 15 min at RT using reblot plus strong antibody stripping solution (Millipore, 60512), the membranes they were incubated with mouse monoclonal anti-ACTB (Millipore Cat #MAB1501, RRID: AB_2223041, 1/10,000) ON at 4° C. and revealed with anti-mouse IgG coupled to peroxidase (Jackson Immunoresearch, 1/10,000). Filter binding assay was performed as previously described (Cronin et al., 2010). The human brain specimens were provided by NeuroCEB. After dissection of the brain of 18-month Nxnl2^{+/+} an Nxnl2^{-/-}, the cerebellum was removed. Brain extracts were made in lysis buffer (10 mM Tris HCl, pH 8.0, 150 mM NaCl, 1 mM EDTA, 1% NP40, 1% sodium deoxycholate), sonicated and suspended in PBS 2% SDS. 50 μg of protein extract was filtered through 0.22 μm nitrocellulose membrane using Bio-Dot SF assembly (Bio-Rad, Hertfordshire, UK). The 0.22 μm membrane was probed with mouse monoclonal anti-TAU antibody (Santa Cruz Biotechnology Cat #sc-58860, RRID: AB_785931, 1/500). To study TAU expression, oligomerization and phosphorylation, extracts from 18-month Nxnl2^{+/+}, Nxnl2^{-/-} (untreated and treated), Mapt^{+/+} and Mapt^{-/ -} brains without cerebellum were sonicated twice 10 s on ice in 50 mM Tris-HCl (pH 7.5), 1 mM EDTA, 1 mM DTT, 1% Triton X-100, 1 mM PMSF, 0.14 mM TLCK, a cocktail of proteinase inhibitors (P2714, Sigma) and phosphatase inhibitor cocktail (524627, Calbiochem). After 30 min on ice, the extracts were centrifuged 5 min at 12,000 g at 4° C. and 40 μg of the supernatant, the whole cell extracted were loaded with Laemmli buffer on a SDS-gel, then transferred to a 0.45 mm polyvinylidene fluoride (PVDF) membrane (Millipore). For oligomerization, a 4-12% Bis-Tris protein gel (ThermoFisher, Cat #NP0322BOX) was used instead of SDS-gel that was run under non-reducing conditions. After saturation, the membranes were incubated either with chicken polyclonal antibodies anti-GFP (Abcam Cat # ab13970, RRID: AB_300798, 1/5,000) mouse monoclonal anti-TAU antibody (Santa Cruz Biotechnology Cat #sc-58860, RRID: AB_785931, 1/500), mouse monoclonal anti-phosphoTAU^{AT100} antibody (Thermo Fisher Scientific Cat #MN1060, RRID: AB_223652, 1/200 or mouse monoclonal anti-phosphoTAU^{AT8} antibody (Thermo Fisher Scientific Cat # MN1020, RRID: AB_223647, 1/200) ON at 4° C. Western blots were revealed with anti-species IgG coupled to peroxidase (Jackson Immunoresearch, 1/15,000). After stripping as above, the membranes were incubated with mouse monoclonal anti-ACTB (Millipore Cat #MAB1501, RRID: AB_2223041, 1/10,000) ON at 4° C. and revealed with anti-mouse IgG coupled to peroxidase (Jackson Immunoresearch, 1/10,000). The quantification was made by respecting the proportion of bands in each piece of membrane assembled as indicated by a chevron (<). Extracts from 2-month Nxnl2^{+/+} and Nxnl2^{-/-} brains without cerebellum were prepared and analyzed with mouse monoclonal anti-phosphoTAU^{AT100} antibody (Thermo Fisher Scientific Cat #MN1060, RRID: AB_223652, 1/200) as above. After stripping, the membrane was incubated with mouse monoclonal anti-ACTB (Millipore Cat #MAB1501, RRID: AB_2223041, 1/10,000) ON at 4° C. and revealed with anti-mouse IgG coupled to peroxidase (Jackson Immunoresearch, 1/10,000). The membrane was then re-stripped and analyzed for the absence of signal using the secondary antibody alone, then incubated with mouse monoclonal anti- GFAP (Sigma-Aldrich Cat #G3893, RRID: AB_477010, 1/1,000).

### Results

The Behavior of the Mouse with a Targeted Inactivation of the Nucleoredoxin-like 2 Gene is Syndromic

We initially observed that Nxnl2^{-/-} mice were hyperactive as compared to Nxnl2^{+/+} wild type controls, but also to Nxnl1^{-/-} mice created on the same genetic background using the same technology. We performed a global behavioral analysis, reported here ordered by test sessions, using two groups of 12 ♂ mice at 2 months of age (data not shown). The use of only one sex was justified by the objective of getting more homogenous results and does not reflect any presumed biological gender distinction (Clayton and Collins, 2014). The animals were monitored for spontaneous water and food intake and rear activity during three sequential phases, habituation, dark and light. Nxnl2^{+/+} mice display an expected nocturnal drinking activity that is perturbed for Nxnl2^{-/-} mice (data not shown). During lightning periods, Nxnl2^{-/-} are drinking more often than Nxnl2^{+/+} mice, but this is the contrary in the dark period (data not shown A). A similar situation was observed for their feeding behavior, but Nxnl2^{-/-} mice do not exhibit reduced feeding at night. In fact, over a 32-hour testing period, Nxnl2^{-/-} mice have a higher food consumption (data not shown). The number of rears that scores the exploratory behavior is also perturbed for Nxnl2^{-/-} mice which display increased vertical activity during the 32-h testing period (data not shown). The disorganization of the circadian activity of mice lacking the Nxnl2 gene is likely resulting from the loss of expression of the gene in the pineal gland, an indirectly light-sensitive part of the circadian system that harbors photoreceptor-related pinealocytes (Wolloscheck et al., 2015). Daily profiling of Nxnl2 gene expression in the pineal gland shows a higher level of expression during daylight. The pineal gland is a crucial structure of the circadian system that is connected to the suprachiasmatic nuclei, the central circadian clock in mammals (Satishchandra and Mathew, 2008). Overall, hyperphagia occurs in the absence of weight gain for Nxnl2^{-/-} mice (data not shown), suggesting that it is a consequence of their higher rearing activity and increased general activity (Ellacott et al., 2010).

This is visible in the open-field test performed with 150 1x in the center of the arena. The distance traveled is longer for Nxnl2^{-/-} mice in 5 min test sessions carried in a novel open field (data not shown), and is overall longer (p=0.0078). A representative path trace of the trajectories shows that the Nxnl2^{+/+} mouse explores more often the periphery of the arena, while the Nxnl2^{-/-} mouse crosses many times the center surface (data not shown). While the traveled distance may translate to a higher general activity of Nxnl2^{-/-} mice in agreement with the hyperactivity observed initially, the lack of aversion for brightly lit, open and unknown environments of the Nxnl2^{-/-} mice shows that these animals have lost, at least partially, a phylogenetic anxiety-inhibitory behavior (La-Vu et al., 2020) (data not shown).

The animals compared have similar body weight (data not shown) and locomotor activity (data not shown), but Nxnl2^{-/-} mice have an average body temperature of 37.75° C., higher to that of 37.31° C. of Nxnl2^{+/+} mice (data not shown). The core body temperature is affected by time of the day as manifested through the circadian temperature rhythm (Gordon, 2017). In the mouse, body temperature is controlled by circadian lipid metabolism by thermogenic brown adipose tissue whose mitochondrial uncoupling increases energy expenditure under cold-stressed conditions (Adlanmerini et al., 2019). During daytime, the mouse prefers an ambient temperature that is just 4-6° C. below its core temperature, and consequently behavior tests, performed here at 21-22° C. are done under slightly cold-stressed conditions (Fischer et al., 2018). The temperature of Nxnl2^{-/-} mice was measured during the day, when they are abnormally active (data not shown), which can explain the difference in core body temperature with Nxnl2^{+/+} mice. The high temperature is a possible consequence of its measurement during higher activity periods since brown adipose tissue metabolism is increased by both cold exposure and exercise (Gaspar et al., 2021; Rodrigues et al., 2018), or by psychological stress-induced hyperthermia (Kataoka et al., 2014).

Compared mice have similar muscular strength (data not shown), but Nxnl2^{-/-} mice have a shorter mean latency in the string test (data not shown). This traction reflex relies on the coordination between forelimb-hanging to gain hindlimb traction. The reduced latency shows that Nxnl2^{-/-} mice have an over operating anteroposterior motor coordination by the cerebellum (Sakayori et al., 2019). This is supported by the higher performance of Nxnl2^{-/-} mice in a test that measures the ability of an animal to maintain balance on a rotating rod (data not shown). This task requires motor coordination controlled by the cerebellum with many other regions involved in proprioceptive and vestibular functions. The mean latency before falling in the rotarod test is not correlated (r=0.1157, n=24) to the total distance traveled in the open field, indicating motor skills of the Nxnl2^{-/-} mice were not acquired by increased locomotion.

The spatial and memory performance of Nxnl2^{-/-} mice were first tested using Y-maze under 100 1x of light. The number of arm entries of Nxnl2^{-/-} mice is higher than that of Nxnl2^{+/+} mice (data not shown), which correlates with a higher locomotor activity. The inactivation of the paralogue gene Nxnl1, whose expression is restricted to the retina, does not trigger this phenotype (data not shown). The specific task that relies on spatial working memory is the natural tendency to choose an alternative arm over an arm previously explored what is scored as % of spontaneous alternation (Webster et al., 2014). We observed a non-statistical trend that was confirmed to be statistically significant by adding a second cohort (data not shown). The Nxnl2^{-/-} mice do not remember correctly which arm they have previously visited, which implies a deficit in learning and memory.

We alternated working memory tests with a test of anxiety-related behavior for logistic reasons dictated by the workflow. The tail suspension test is widely used to evaluate the antidepressant-like effects of drugs (Stukalin et al., 2020). The decrease in the duration of the immobility shows that Nxnl2^{-/-} mice have a reduced susceptibility to despair (data not shown), inversely correlated (r=−0.5893, p=0.0024) to the latency of the first immobilization (data not shown). This invert correlation improves the detection of antidepressant-like behavior of Nxnl2^{-/-} mice (Castagne et al., 2009) and confirms the decrease in anxiety-related behavior previously observed (data not shown).

The amplitude (arbitrary units) of the acoustic startle reflex of Nxnl2^{-/-} mice to a startling acoustic pulse of 110 dB, but not for prepulses with lower intensities (70, 80, 85 and 90 dB/10 ms) is reduced as compared to Nxnl2^{+/+} mice (data not shown). When a low-salience auditory stimulus precedes an unexpected startle-like acoustic stimulus, the startle motor reaction becomes less pronounced (Gomez-Nieto et al., 2020). This phenomenon, known as prepulse inhibition, is normal for Nxnl2^{-/-} mice (data not shown). The deficit of acoustic startle reflex of Nxnl2^{-/-} mice could translate the anxiolytic effect of the inactivation of Nxnl2 gene (Hur et al., 2020) (data not shown). Alternatively, since aged Nxnl2^{-/-} mice have a deficit in vision and olfaction (Jaillard et al., 2012), we cannot rule out that the reduced amplitude of the acoustic startle reflex is translating a reduction of auditory function, even at 2 months of age.

The associative and memory performance of Nxnl2^{-/-} mice were then tested after habituation to sessions during which a stimulus (the cue, a tone [symbol] ) is paired with an aversive unconditioned stimulus (an electric foot-shock) (Aincy et al., 2018). Nxnl2^{+/+} mice respond to the aversive stimulus by reducing their locomotor activity more than Nxnl2^{-/-} mice data not shown). The following day, the percentage of freezing of Nxnl2^{-/-} mice when replaced the same environmental context ( [symbol] ) is lower than Nxnl2^{-/-} mice (data not shown). The percentage of freezing of Nxnl2^{-/-} mice is also lower than Nxnl2^{+/+} mice in response to the cue ( [symbol] ) (data not shown). This points to a dysfunction of a neural circuit involving the amygdala, the cerebral cortex and the hippocampus (Crawley, 2007). This prompted us to look at the response to acute thermal pain of the animals that may interfere with the learning and memory test of cued and contextual fear conditioning. A heat stimulus applied to the tail does not trigger a difference in the response between the two mouse genotypes (data not shown). Nevertheless, the first reaction of Nxnl2^{-/-} mice (licking/jumping) on a 52° C. plate is delayed compared to Nxnl2^{+/+} mice (data not shown) indicating the nociceptive threshold is abnormally high for Nxnl2^{-/-} mice. Mice exhibit a marked fear of novel stimuli (Wilson and Mogil, 2001). Pain and anxiety are closely linked and the reduction of anxiety is accompanied by a parallel decrease in pain sensitivity (Zhang et al., 2014). The reduced latency of Nxnl2^{-/-} mice in the hot-plate test is likely due to their anxiolytic-related behavior (data not shown).

Intraperitoneal injection of 50 mg/kg of pentylenetetrazol (PTZ), a convulsant drug, triggers an extended latency of Nxnl2^{-/-} mice compared to wild-type controls (data not shown), but the seizure profiles are comparable. One should notice that similar antiseizure effects of PTZ-induced seizures were observed after systemic administration of fructose-1,6-bisphosphate (FBP) in vivo (Lian et al., 2007). Epilepsies, characterized by convulsive frequent febrile seizures are linked to impaired brain glucose metabolism (Greene et al., 2003). Glucose transporter type 1 deficiency syndrome (GLUT1DS) causes epilepsy, movement disorders, and cognitive impairment (Schwantje et al., 2020). This points to a modification of the brain glucose metabolism generated by the inactivation of the Nxnl2 gene.

Using unique cohorts allows the analysis of correlations. So, the anxiolytic-related behavior (anti-anxiety) measured during open field tests is correlated to the depression-like behavior seen by tail suspension immobility duration (data not shown), which is inversely correlated to pain sensitivity measured by the hot-plate tests (data not shown). Pain sensitivity is inversely correlated to sensorimotor gating, measured by acoustic startle reactivity (r=−0.4759, p=0.0187) and to sensorimotor abilities, measured by string tests (r=−0.4993, p=0.0180). Sensorimotor abilities (string tests) are correlated to PTZ sensibility (r=0.4518, 0.0455, n=11) and inversely correlated to core body temperature (r=−0.5132, p=0.0146). Sensorimotor abilities measured by the rotarod test is correlated to pain sensitivity measured by the hot-plate test (r=0.4176, p=0.0423). Learning and memory measured by spontaneous alternation in the Y-maze is inversely correlated to sensorimotor abilities tested in the rotarod test (r=−0.4728, p=0.0263). The inactivation of the Nxnl2 gene triggers a complex syndrome in which fear, pain sensitivity, coordination, learning and memory and possibly brain glucose metabolism are deficient what could be translated by an abnormally high core body temperature. The anxiolytic effect is regulated by the amygdala that is connected to the temporal two-thirds of the distal portion of hippocampal Cornu Ammonis (CA)1 region (Andersen et al., 2006; Jimenez et al., 2018).

To address learning and memory in a test that does not depend on pain sensitivity, we used a novel cohort of ♂ to test the animals under 100 1x of ambient light in the Morris water maze at water temperature of 20-21° C. After training, the latency to reach the visible platform of the Nxnl2^{-/-} mice is longer than for the Nxnl2^{+/+} control mice (data not shown). When the platform was hidden under the surface of water, a deficit in latency was also observed for Nxnl2^{-/-} mice (data not shown). Mice of both genotypes ameliorate every day their performance in the test using either visible or hidden platform, but while the deficit of Nxnl2^{-/-} mice is observed from the first day with the visible, platform, it is only perceptible at day two and statistically significant at day three with the hidden platform. The performances of this test rely on hippocampal-dependent visuospatial navigation (Medlej et al., 2019). The vision of Nxnl2^{-/-} mice starts to deteriorate only after two months of age which cannot impairs with the test performed here on 2-month animals (Jaillard et al., 2012).

### The Inactivation of the Nucleoredoxin-like 2 Gene Impairs the Function of the Hippocampus

The Nxnl2^{-/-} syndrome was addressed in regard to synaptic plasticity. To test Nxnl2^{-/-} mouse by electrophysiology, we measured field excitatory postsynaptic potentials (fEPSP) with glass microelectrodes placed in the CA1 stratum radiatum of ex vivo prepared hippocampal slides and recorded fEPSP before and 40 min after high frequency stimulation (HFS) (Le Roux et al., 2013). The traces of the postsynaptic recording are distinct between the two genotypes, but not that of presynaptic recording (data not shown). When normalized, the slopes of signals of Nxnl2^{-/-} hippocampal slides does not emerge from that of Nxnl2^{+/+} after HFS (data not shown). The 50-60 min period of the recording shows a deficit in the persistent enhancement of neurotransmission following HSF or long term potentiation (LTP) of Nxnl2^{-/-} hippocampus (data not shown) (Kerchner and Nicoll, 2008). LTP is a form of synaptic plasticity that is most closely linked to memory storage, so that the electrophysiological data support the deficit of Nxnl2^{-/-} mice in the learning and memory tasks (Abraham et al., 2019).

We repeated the analysis of LTP by comparing that of the Nxnl2^{-/-} mouse to an additional mouse model constructed by inserting a β-galactosidase reporter within the Nxnl2 locus (data not shown D). The CA1 basal synaptic transmission from slides of the Nxnl2^{R/R} hippocampal was not altered compared to Nxnl2^{+/+} hippocampus (data not shown). Likewise, CA1 basal synaptic transmission does not differ between Nxnl2^{-/-} and Nxnl2^{+/+} hippocampus (data not shown). The Nxnl2^{-/-} was constructed by deleting exon 1, encoding for RdCVF2, by homologous recombination (Jaillard et al., 2012) (data not shown). For the homozygous reporter mice (Nxnl2^{R/R}), the introduction of the reporter cassette erases the sequence of both RdCVF2 and RdCVF2L (data not shown). The trace of the postsynaptic recording shows a deficiency of Nxnl2^{-/-} versus Nxnl2^{+/+} hippocampus (data not shown) and as well as for Nxnl2^{R/R} versus Nxnl2^{+/+} hippocampus (data not shown). We recorded a deficit of fEPSP, expressed as % as a baseline, for the Nxnl2^{-/-} hippocampus as previously (data not shown) and for the Nxnl2^{R/R} hippocampus (data not shown). Quantitatively, for the 40-60 min period, LTP are lower for the Nxnl2^{-/-} (data not shown), and the Nxnl2^{R/R} hippocampus (data not shown). The two mouse models of Nxnl2 inactivation were produced in distinct genetic backgrounds (BALB/c and C57B/L6N), which reinforce the argument that the deficit is linked to the inactivation of the Nxnl2 gene. Recordings, performed in parallel, reveal no quantifiable influence of the genetic background on LTP generated by the hippocampus.

### The Nucleoredoxin-like 2 Gene is Expressed in the Area Postrema

A corollary to the deficit in brain function of the Nxnl2^{-/-} is that the Nxnl2 gene must be expressed in the brain. For the reporter allele R, β-galactosidase expression is presumably under the control of the endogenous Nxnl2 promoter, located in 5′ on its open reading frame, as shown in the retina (Lambard et al., 2010). In this configuration, the reporter will not distinguish the expression of RdCVF2L from that of RdCVF2, the later resulting from intron retention. Nevertheless, β-galactosidase staining of mouse tissues indicates the regionalization of Nxnl2 expression, taken as a whole. As expected from a previous study, the Nxnl2^{R/+} mouse at 2 months showed signals in the olfactory tube (Jaillard et al., 2012) (data not shown). At higher resolution, the staining can be delineated to the olfactory sensitive neurons (data not shown). These receptor neurons project their axons to the glomerular layer of the olfactory bulb (data not shown). The adequacy between the β-galactosidase staining pattern and what is known of Nxnl2 expression confirms that the Nxnl2^{R/+} mouse is an appropriate model to explore Nxnl2 expression in the brain. We sectioned the brain of a Nxnl2^{R/+} mouse at 2 months to reveal the endogenous expression of the Nxnl2 gene at that age. We compared the blue coloration of stained sections of thirty-five regions of the Nxnl2^{R/+} brain to that of the negative control (Nxnl2^{+/+}, C57BL/6N), both at 2-month of age. We detected scattered signals mapping to regions involved in the behavior that is altered in the Nxnl2^{-/-} mouse, such as the hippocampic formation, the central, lateral, basolateral and basomedial nuclei of the amygdala, and throughout many other brain regions: EBRAINS—Expression profile of the nucleoredoxin-like 2 gene in the mouse brain using a beta-galactosidase knock-in reporter strain (Léveillard et al., 2021). For example, Nxnl2 expression in the subiculum which is the main hippocampal exit through afferent ways from CA1. The most prominent and ordered signal was observed in the area postrema (data not shown). The expression of the reporter protein was restricted to a subset of cells of area postrema (Price et al., 2008) (data not shown). The area postrema is a member of the circumventricular organs composed of fenestrated capillaries with discontinuous expression of tight junction and extensive interactions of parenchymal cells of this organ with the cerebrospinal fluid (CSF) and blood circulation (Wang et al., 2008). The reporter signal is increased in the area postrema of the Nxnl2^{R/R} mouse, which indicates that the survival of Nxnl2 expressing cells of the area postrema does not require the action of the Nxnl2 gene, at least up to 2 months (data not shown). Similar observations were made for another circumventricular organ, such as the subfornical organ involved in thirst and hunger (Mckinley et al., 2019) (data not shown). The sensory subfornical organ in the forebrain, as the area postrema in the hindbrain, lacks a normal blood-brain barrier such that neurons, within them, are exposed to blood-borne agents. This is also true for an adjacent positive region, the median preoptic nucleus that is involved in core body thermoregulation (da Conceicao et al., 2020) (data not shown). Neurons in the median preoptic nucleus receive afferents from the subfornical organ.

The expression of Nxnl2 is circumscribed, but not restricted, to regions of the brain that are permeable to blood-borne molecules such as circulating hormones. The proximity of the Nxnl2 expressing cells in the area postrema to microvascular can be appreciated by immunohistochemistry using antibody against plasmalemma vesicle-associated protein (PLVAP/MECA-32) (data not shown). MECA-32 is expressed in central and peripheral vasculature throughout development, but its expression in the cerebrovasculature is downregulated upon the establishment of the blood-brain barrier in the adult, remaining only expressed in vascular endothelial cells that establish fenestrated capillaries (Yu et al., 2012). By RT-PCR, we show that the mRNA of both products of the Nxnl2 gene, the trophic factor RdCVF2 and the thioredoxin-related protein RdCVF2L are expressed by cells of the area postrema of the Nxnl2^{+/+} mouse and confirm the absence of the RdCVF2 and RdCVF2L mRNAs in the homozygous Nxnl2^{R/R} mouse (data not shown). Since RdCVF2 is secreted (Chalmel et al., 2007) and because the action of its paralogue, RdCVF, is relayed by the glucose transporter GLUT1 (Ait-Ali et al., 2015), we looked at the expression of GLUT1 in the area postrema of the Nxnl2^{+/+} mouse. The expression of GLUT1 excluded a local role of GLUT1 in the potential protective action of RdCVF2, as expected (Maolood and Meister, 2009) (data not shown).

### Impaired Metabolism of Hippocampal Specimens of Nxnl2^{-/-} Mice

In rodents, the area postrema is a single structure that descends out in to the 4^{th} ventricle. By its position, even in the presence of an ependymal layer along the ventricular walls of the area postrema (Kiecker, 2018), the signals generated in the area postrema could circulate in the CSF to reach the brain areas that participate in the complex behavioral syndrome of the Nxnl2^{-/-} mouse. The absence of suitable RdCVF2 antibodies led us to test this hypothesis by quantifying the metabolism of the hippocampus, since the paralog RdCVF in the retina regulates retinal metabolism. We standardized the dissection of well-mapped hippocampal specimens of 2 mm in diameter and 0.5 mm in thickness (data not shown). The concentration of 39 metabolites covering 11 metabolic pathways was quantified in quadruplicates pools made of three standard specimens of the hippocampus of 2-month Nxnl2^{+/+} and Nxnl2^{-/-} mice, for two successive experiments using slightly different metabolomic technologies (data not shown). Focusing here on differences in concentrations that are statistically significant for the first experiment, we organized the results centering on glucose consumption, as it is the major source of energy for neurons. The concentration of glucose-1-phosphate, a metabolite of glycogenolysis that corresponds to the production of glucose-6-phosphate (G6P) from glycogen storage, is lower in hippocampus specimens of Nxnl2^{-/-} mice (data not shown). Mice lacking glycogen synthase, essential for glycogen production, have impaired hippocampal LTP, similar to Nxnl2^{-/-} mice (Duran et al., 2013). The concentration of three metabolites of glycolysis: G6P, fructose 1,6-bisphosphate (FBP) and 2/3-phosphoglycerate (2/3PG) is higher in hippocampus specimens of Nxnl2^{-/-} than that of Nxnl2^{+/+} mice (data not shown). The concentration of UDP-N-acetylglucosamine is lower in Nxnl2^{-/-} hippocampus specimens (data not shown). This metabolite is involved in O-GlcNAcylation of targeted proteins and produced by the hexosamine pathway that branches from glycolysis at the level of fructose-6-phosphate (F6P) (Chandel, 2015). O-GlcNAcylation of hippocampal proteins is reduced in brain starving of glucose, which decreases neuronal O-GlcNAcylation level in the hippocampus, impairs cognition and reduces dendritic spine density in the hippocampus of adult mice (Dos Santos et al., 2018; Yang et al., 2017). The concentration of phosphoribosylpyrophosphate, produced by the PPP is also lower in Nxnl2^{-/-} hippocampal specimens (data not shown). Collectively, the results represent an increase in the concentration of glycolytic metabolites and a decrease in that of glycogenolysis, hexosamine pathway and PPP (data not shown). The concentrations of variable glycolytic metabolites are correlated, such as G6P with ⅔PG (r=0.7372, p=0.0150, n=10) and with FBP (data not shown). In order to test the robustness of these data, we repeated the experiment with an additional cohort using an Orbitrap, a more precise instrument (Hu et al., 2005) (data not shown). The additional results confirm the elevated concentration of the three identified glycolytic metabolites in the Nxnl2^{-/-} hippocampal specimens and reveal a fourth one, phosphoenolpyruvate (data not shown). Since both experiments include an internal standard (Wu et al., 2005), we combined their results, revealing that the lower concentration of UDP-N-acetylglucosamine in the Nxnl2^{-/-} specimens is associated with an increase of UDP in the hexosamine pathway, two phenomenon possibly linked metabolically (Love and Hanover, 2005) (data not shown). We found a slight but significant difference between the two experiments by analyzing the ratio ADP/ATP, but that small difference does not modify the general interpretation of the results (Bradbury et al., 2000) (data not shown).

The steady-state concentration of a metabolite is proportional to its enzymatic production and use by the following metabolic reaction. It is consequently impossible to ensure that the increase in the concentration of G6P, as it is a central metabolite in different metabolic pathways, result from a higher rate of its synthesis by hexokinase or a reduced rate of entry into PPP, glycogen synthesis or glycolysis (Chandel, 2015). Since the conversion of glucose to G6P is irreversible (Camacho et al., 2019), the production of G6P from glucose is directly linked to intercellular glucose that is uptaken by cells of the central nervous system by facilitative diffusion glucose transporters of the SLC2A family (Koepsell, 2020). We analyzed the expression of the three major SLC2A glucose transporters, GLUT1, GLUT3 and GLUT4 in the standard specimens of the hippocampus by western blotting. The expression of GLUT1 was found to be equivalent for both genotypes (data not shown). A similar observation was made for GLUT3 (data not shown). We detected two isoforms of the insulin-responsive glucose transporter GLUT4, the full-length isoform migrating above 50 kDa and a second isoform, most likely an non-functional GLUT4 protein (ΔEx-3-5) resulting from alternative splicing (Buchner et al., 2015) (data not shown). The expression of the functional and full-length isoform is specifically reduced in the hippocampal specimens of Nxnl2^{-/-} mice, which could contribute to the deficit in learning and memory of these mice since insulin modulates hippocampus-mediated spatial working memory via GLUT4 (McNay and Pearson-Leary, 2020) (data not shown). The expression of glucose transporters and levels of metabolized analyzed by metabolomics suggests that the accumulation of glycolytic metabolites in the Nxnl2^{-/-} hippocampus results in the deceleration of the metabolic flux triggering metabolite accumulation, captured at the time of the sacrifice of the animals.

### The Synergistic Action of RdCVF2 and RdCVF2L in Repairing the Hippocampal Dysfunction of Nxnl2^{-/-} Mice

The Nxnl2^{-/-} hippocampus metabolic profiling supports that by analogy to RdCVF, produced by rods to stimulates cone metabolism in the retina (Leveillard and Sahel, 2017), RdCVF2 produced in the area postrema increases glycolysis in cells within the hippocampus. In order to test this hypothesis, we constructed self-complementary adeno-associated vectors (AAV) encoding for RdCVF2 or RdCVF2L and GFP via a self-cleaving 2A peptide under the control of the cytomegalovirus enhancer/chicken β-actin (CMV/CBA) promoter (Vasireddy et al., 2013). Serotype 9 allows AAV vectors to penetrate the brain when injected into the bloodstream of neonatal mice before the establishment of the blood-brain barrier (Ait-Ali et al., 2015; Dalkara et al., 2012). In order to account for the purity and the functional titer of the AAV particles, we characterized the viral preparations with silver stain gel and electron microscopy. The silver stain showed no impurities other than the VP1-3 proteins in expected ratios (Naso et al., 2017) (data not shown). The percentage of empty capsid particles of these preparations was quantified by transmission electron microscopy after uranyl acetate staining (Grieger et al., 2016) (data not shown). While, the negative control (AAV2/9-GFP) has a ratio empty/total distinct from the two other recombinant AAVs, these two preparations have an indistinguishable ratio.

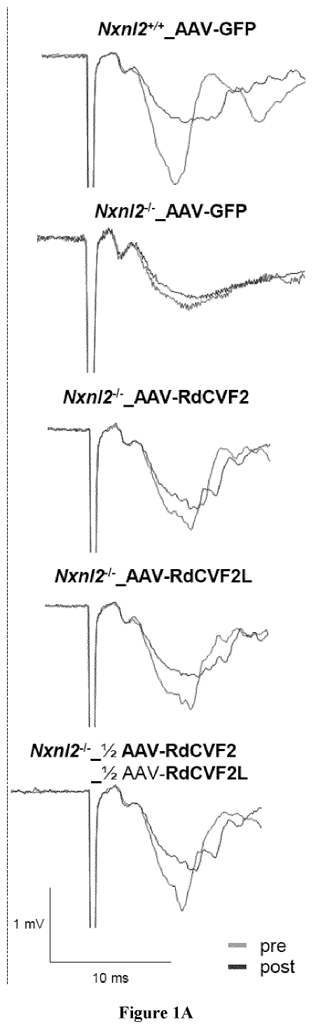

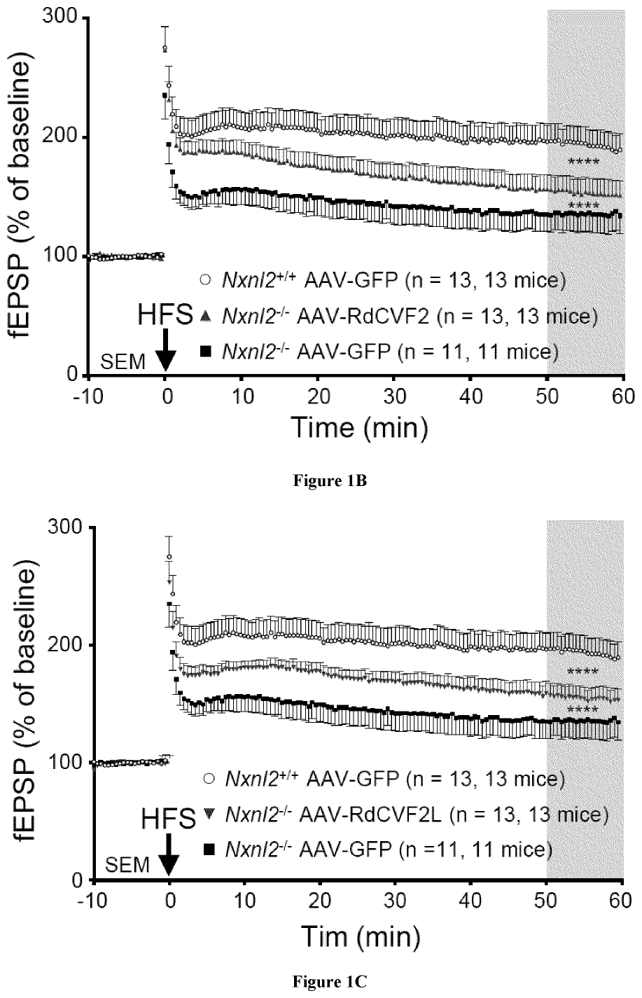

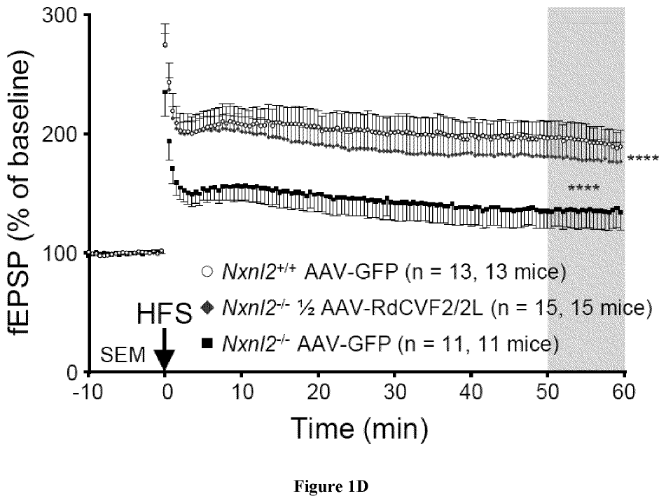

Following the intracardiac injection of Nxnl2^{-/-} ♂ mice at post-natal day (PN) 4, the distribution of the transgene in the brain at 2 months was examined using anti-GFP immunohistochemistry (data not shown). Among the cells transduced by AAV2/9.2YF-CMV/CBA-RdCVF2^{2A}GFP the pyramidal neurons of the hippocampus are predominant (data not shown). This peculiar tropism is not dependent on the encoded sequence since it was also observed for AAV2/9.2YF-CMV/CBA-RdCVF2L^{2A}GFP, as seen in a previous study (Aschauer et al., 2013) (data not shown). CA1 basal synaptic transmission of the Nxnl2^{-/-} hippocampus is slightly higher than that of Nxnl2^{+/+} at two months after administration of the negative control (data not shown). A similar observation was made for the two other vectors delivered individually or in combination (data not shown). Nevertheless, no difference in the CA1 basal synaptic transmission could be observed between Nxnl2^{-/-} at 2 months after delivery of RdCVF2 or RdCVF2L encoding AAVs (data not shown). For measuring LTP, we proceeded as previously except we recorded only one hippocampal slide per mouse to assure sphericity, which permits the use of two-way ANOVA—repeated measures for the statistical analysis of the results (Bate and Clark, 2014). At two months, the traces of postsynaptic recordings show an alteration for Nxnl2^{-/-} versus Nxnl2^{+/+} hippocampus after AAV-GFP injection and intermediate situation for the Nxnl2^{-/-} mice injected with Nxnl2 gene products (FIG. 1A). RdCVF2 alone rescues partially but significantly the fEPSP response of the Nxnl2^{-/-} hippocampus when compared to AAV-GFP, supporting the formulated hypothesis above (FIG. 1B). Interestingly, we observed the same partial correction after the injection of AAV-RdCVF2L (FIG. 1C). More importantly, the combined administration of half the dose of RdCVF2 and RdCVF2L results in an almost complete reversion of LTP deficit of Nxnl2^{-/-} mice at 2 months (FIG. 1D). This indicates that both products of the Nxnl2 gene act synergistically to restore the altered function of the hippocampus of the Nxnl2^{-/-} mouse. A close view of the fEPSP measures immediately following the HSP reveals a distinct recording traces that may be related to two different but coordinated rescue mechanisms (data not shown). Following our hypothesis, the non-cell-autonomous activity of RdCVF2 does not require that AAV-RdCVF2 targets any specific region of the brain. However, the presumably cell-autonomous action of the thioredoxin-related protein RdCVF2 requires the transduction of dysfunctional cells, most likely, the hippocampal pyramidal neurons (data not shown).

We analyzed the effect of the corrective therapy on cellular metabolism using 2-month hippocampal specimens (data not shown). Overall, the intracardiac injection of AAV does not modify the metabolism within the hippocampus, since the concentrations of metabolites are equivalent in Nxnl2^{+/+} and Nxnl2^{-/-} mice injected with AAV-GFP to those without injection (data not shown). The three glycolytic metabolites (G6P, FBP and ⅔PG) are elevated in the Nxnl2^{-/-} specimens after injection of either AAV-GFP or ½ AAV-RdCVF2+½ AAV-RdCVF2L (data not shown). As previously observed, the concentration of G6P and FBP are correlated (data not shown). The glycogenolysis (data not shown) and the hexosamine pathway (data not shown) do not seem to be modified. Surprisingly the PPP was affected, as previously but was not corrected by the products of the Nxnl2 gene (data not shown). We could not identify any metabolite that correlates statistically with the corrective effect measured by electrophysiology. We have used in that experiment animals of both sexes, but this is not the reason for the lack of effect on the metabolism (data not shown). Pyramidal neurons that generate LTP represent a subset of the cell population and even possibly involve only a subset of synapses drowned in the mass of the hippocampus specimens (Keller et al., 2018). This situation may preclude the identification to any significant change in metabolite concentration in the hippocampus as a whole. We also analyzed the level of expression of GLUT4 in hippocampal specimens after AAV delivery. The treatment did not restore GLUT4 expression, but the result can be interpreted as a trend (data not shown). This observation may be explained by the fact that only a subset of cells expresses GLUT4, among which the pyramidal neurons (Ashrafi et al., 2017). We validated the transduction of the AAVs expression of GFP in these specimens (data not shown).

### TAU Becomes Aggregated in the Brain of the Nxnl2^{-/-} Mouse by 18 Months of Age

The contribution of RdCVF2L in the reversion of deficit in LTP was explored in the light of its ability to prevent TAU phosphorylation (Elachouri et al., 2015). TAU phosphorylation favors its aggregation leading to the formation of NFT (Gyparaki et al., 2021). At 2 months, the absence of difference in the status of TAU phosphorylation in the brain of Nxnl2^{-/-} versus Nxnl2^{+/+} mice, rules out the idea that LTP deficit of the Nxnl2^{-/-} mice produced by interference of TAU aggregation. (Wesseling et al., 2020) (data not shown). But since the phosphorylation and the aggregation in the retina was observed in condition of oxidative stress, which is a hallmark of aging, we analyzed TAU post-translational modification and its aggregation in the brain of aged Nxnl2^{-/-} mice. First, we discovered that the brain of the Nxnl2^{-/-} mice show signs of neuroinflammatory astrogliosis at 10 months (data not shown). The upregulation of glial fibrillary acidic protein (GFAP) is likely preceded by the polarization of microglia (resident macrophages) (Ullah et al., 2020) that would aggravated hippocampal dysfunction by reducing glucose availability for neurons, as activated microglial cells shift towards glycolysis during inflammation (Afridi et al., 2020). Nevertheless, at 2 months, the Nxnl2^{-/-} brain does not display any elevated level of expression of the neuroinflammatory marker, GFAP. Consequently, LTP deficit at 2 months does not the result from neuroinflammatory astrogliosis (data not shown).

By 18 months of age, TAU aggregation was found to be elevated in the brain of the Nxnl2^{-/-} mice using filter finding assay on the whole brain (data not shown) (Cronin et al., 2010; Nanavaty et al., 2017). Human brain specimens from age-matched patients without and with NFT observed by anatomical pathology validate the assay (Braak and Braak, 1991). The expression of TAU protein is not modified in these conditions (data not shown). The absence of expression of TAU in the brain specimen of the Mapt^{-/-} mouse demonstrates that the signal detected by western blotting is specific (Dawson et al., 2001). TAU oligomers were more abundant in the brain of Nxnl2^{-/-} mice (data not shown). They are probably composed of phosphorylated TAU proteins, as seen in brain specimens of AD patients (Maeda et al., 2006). A growing body of evidence indicates that TAU oligomer formation precedes the appearance of NFT and contributes to neuronal loss. Cysteines residues (C⁶⁰⁸ and C⁶³⁹, P10636-1) within the regions R2 and R3 of the microtubule binding domain of TAU are involved in the formation of these oligomers of TAU (Soeda et al., 2015). Phospho-TAU antibody AT100 is specific to the phosphorylated TAU at f T⁵²⁹, S⁵³¹ and T⁵³⁴, AT8 recognizes Ser⁵¹⁹ and T⁵²² (P10636-1) (Malia et al., 2016; Yoshida and Goedert, 2006). The sequence surrounding these phosphorylated residues encompasses 45 amino-acids region that is 100% identical between human and mouse TAU (P10637-1). Those two well-studied epitopes are frequently found in postmortem brain specimens of patients who died of AD (Wesseling et al., 2020). We found more phosphorylation using AT100 (data not shown) and AT8 antibodies (data not shown) in whole brain samples from Nxnl2^{-/-} as compared to Nxnl2^{+/+} mouse brains. We also found correlations of aggregation, oligomerization and phosphorylation. The correlation of aggregation with oligomerization (r=0.6164) is lower than with phosphorylation at both AT100 (r=0.9406) and AT8 (r=0.8889) epitopes, suggesting that TAU oligomerization precedes its phosphorylation in the temporal sequence that leads to its aggregation within the Nxnl2^{-/-} brain (data not shown). Phosphorylation at AT8 and AT100 epitopes are highly correlated (r=0.9119, p<0.0001). We did not map the various stages leading from TAU oligomerization to its aggregation within the Nxnl2^{-/-} brain. In AD brains, the formation of NFT expands from the parahippocampal gyrus to the hippocampus and further to the cortex, so it is quite possible that the hippocampus of the Nxnl2^{-/-} is affected by TAU aggregation (Uchihara, 2020).

### Prevention of TAU Aggregation in the Brain of the Nxnl2^{-/-} Mouse by 18 Months' Gene Therapy

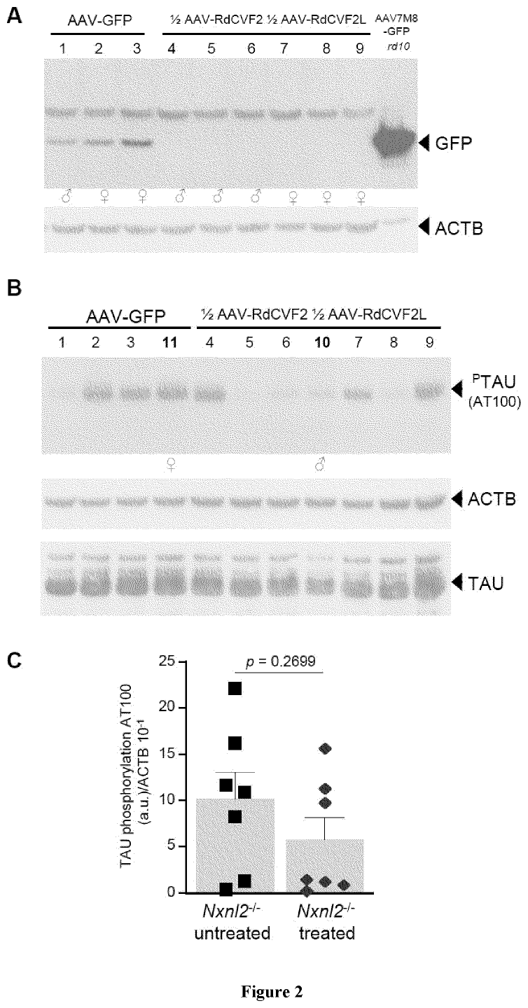

Nxnl2^{-/-} mice got an intracardiac injection of recombinant AAV vectors at PN4, then housed in normal conditions for 18 months. Then, after sacrifice, the expression of the AAV transgene was analyzed by western blotting using 80 μg of whole brain extract with an anti-GFP antibody. As a positive control, we used a retinal extract of an rd10 mouse subretinally injected with an AAV2-7M8-CMV/CBA-GFP (Byrne et al., 2015). We detected the expression of GFP in the brain of all Nxnl2^{-/-} mice injected with AAV2/9-2YF-CMV/CBA-GFP (FIG. 2A). However, we could not detect the expression of GFP in the brain of animals injected with ½ AAV2/9-2YF-RdCVF2^{2A}GFP combined with ½ AAV2/9-2YF-RdCVF2L^{2A}GFP. The absence of detectable GFP is likely reflective of the lower expression of GFP positioned downstream of a self-cleaving 2A peptide, as observed previously in the retina (Ait-Ali et al., 2015; Kim et al., 2011) (data not shown), and the lower dose injected in this experiment (1×10¹² vg) as compared to 4×10¹² vg (data not shown). Bypassing this limitation, we measured semi- quantitatively the phosphorylation of TAU using AT100 antibody. The reduction of TAU phosphorylation in the whole brain of the Nxnl2^{-/-} mice treated with both products of the Nxnl2 gene is striking, even if it not statistically significant (FIG. 2B). There is a 20% reduction of the expression of TAU in those specimens, but this cannot explain the reduction of its phosphorylation. Since it has not been possible to visualize the expression of the transgenes, we do not know if mice 4 and 9 express the corrective genes. By excluding these two mice, 18% of the effective of the cohort, the difference in AT100 phosphorylation, standardized to the expression of TAU, grazes statistical significance (p=0.0719). Excluding in addition mouse 1 that did not develop TAU phosphorylation for unknown reason, 27% of the cohort, the result becomes significant (p=0.0068). This phenomenon is also visible if one takes into account the variability of AT100 phosphorylation observed in the untreated animals at 18 months (data not shown). The average AT100 phosphorylation, normalized by cytoplasmic actin, is lower in the treated Nxnl2^{-/-} mice (FIG. 2C). In other words, the treatment with the combination of RdCVF2 and RdCVF2L over 18 months-period reduces the phosphorylation, and by extension the aggregation of TAU, in five out of seven treated Nxnl2^{-/-} mice.

### Discussion

### Possible Mechanisms of the Synergistic Effect of RdCVF2 and RdCVF2L on Hippocampal Function

The Nxnl2 gene is expressed in various parts of the mouse brain with a prominent expression in the area postrema, where both RdCVF2 and RdCVF2L are expressed. While we have not identified the types of cells that express the gene in this part of the brain, the area postrema is located at the interface of the blood circulation that carries peptidic hormones from the periphery to the central nervous system. In addition, the absence of brain blood barrier between this organ, as for other sensory circumventricular organs expressing Nxnl2, puts the gene at a node between circulating hormones regulating directly or indirectly glycemia, the concentration of circulating glucose and its use by neurons of the brain (Fry et al., 2007; MacDonald et al., 2021). The extracellular truncated trophic factor RdCVF2 can participate in the generation of LTP, recorded on hippocampal slices by acting on glucose uptake (Chalmel et al., 2007). The area postrema is in contact with the CSF secreted by the choroid plexus (Damkier et al., 2013). CSF circulation distributes glucose to cells of the brain through its regulated flow (Fultz et al., 2019). The absence of RdCVF2 is sensed by the abnormal glycolysis measured in the hippocampus of the Nxnl2^{-/-} mouse. The restoration of LTP after delivery of RdCVF2 in this mouse model demonstrates the role of this truncated thioredoxin. To our surprise, we failed to restore the metabolism of the hippocampus by re-expressing the products of the Nxnl2 gene under the control of a ubiquitous CMV/CBA promoter. We know that with this approach, RdCVF2 is expressed at abnormally higher levels in many cells in the brain, which is not a natural situation, regarding both its physiological distribution and its expression level (Lambard et al., 2010). This absence of correlation between function and metabolism means that the Nxnl2 gene does not regulate glucose metabolism globally in the brain and that its effect is restricted to a subset of cells, and even a subset of cells in the hippocampus, such as pyramidal cells that generate the LTP in response to an excitatory signal (Ayhan et al., 2021; Habib et al., 2016). The modification of glycolysis in hippocampus is probably due to metabolic plasticity within the organ, such as astrocytes even if no GFAP reactivity could be observed at 2 months (Ebersole et al., 2021). The reintroduction of RdCVF2 under a ubiquitous promoter would not correct for this metabolic plasticity. GLUT4 expression is restricted by cells with altered function, and is downregulation in the Nxnl2^{-/-} hippocampus is certainly involved (Ashrafi et al., 2017). This fits with the regulation of glycolysis by RdCVF2 via its interaction with a cell- receptor expressed by the hippocampal pyramidal neurons as well as by other neurons involved in the other studied behaviors. This putative cell surface receptor is certainly not BSG1 because its expression is restricted to the retina and the pineal gland (Tokar et al., 2017). We speculate that this receptor is complexed with GLUT4 by analogy with the mode of action of RdCVF, through GLUT1 (Ait-Ali et al., 2015).

The synergistic action of RdCVF2 with RdCVF2L is reminiscent of the action of its paralogue Nxnl1, involved in glucose uptake and in redox homeostasis in the retina (Leveillard and Ait-Ali, 2017; Leveillard and Sahel, 2017). A difference in the mode of action of RdCVF2 and RdCVF2L is reflected by non-contiguous fEPSP traces after gene therapy. Concerning redox homeostasis, the reduction of the concentration of one of the metabolites of the PPP in the first experiment is in agreement with such scenario. Nevertheless, one of the cysteines of the catalytic site of the thioredoxin-related protein RdCVF2 is replaced by a serine in all placental mammals for which the genome sequence is available (Elachouri et al., 2015). Consequently, the RdCVF2L protein does not carry a thioredoxin active site, but that of a monothiol glutaredoxin, as glutaredoxin 3 (Haunhorst et al., 2010). Glutaredoxins reduce S-glutathionylation, of redox sensitive cysteines in proteins. Under oxidative stress conditions, cysteines are non-enzymatically oxidized with the tripeptide glutathione (GSH), one of the most crucial cellular thiol buffers (Ren et al., 2017). The formation of protein-SSG, termed S-glutathionylation, protects protein thiols under oxidative conditions, since it can be reverted by electron transfer (Herrero and de la Torre-Ruiz, 2007). It prevents further oxidations of thiol groups of proteins to sulfenic, sulfinic, and sulfonic acids, the latter oxidation being irreversible (Xiong et al., 2011). The protein S-glutathionylation cycle, initiated under oxidative conditions, is inverted when a reducing environment is restored. Deglutathionylation restores protein function and S-glutathionylated glutaredoxin is then reduced by reduced glutathione (Zhang et al., 2018). Hippocampal-dependent learning and memory functions are peculiarly sensitive to oxidative stress (Huang et al., 2015). The protection of LTP by RdCVF2L was achieved by direct transduction of hippocampal pyramidal neurons with AAV2/9-2YF-RdCVF2L.

The positive role of the Nxnl2 gene on synaptic plasticity and memory is theoretically produced by an effect on the N-methyl-D-aspartate (NMDA) receptors (Abraham et al., 2019; Li and Tsien, 2009). Seven cysteines of NMDA-receptor subunits are regulated by oxidoreduction and could be targeted by the monothiol glutaredoxin activity of RdCVF2L (Aizenman et al., 2020; Lipton et al., 2002). The synergistic effect of RdCVF2 and RdCVF2L would result from the action of RdCVF2 regulation of glucose metabolism on neurons of hippocampal pyramidal and by deglutathionylation of NMDA-receptor by RdCVF2L. Deglutathionylation of NMDA-receptor by RdCVF2L depends on metabolism of glucose by the PPP to generate NADPH (Miller et al., 2018) and with RdCVF2 increasing glucose uptake, the action of RdCVF2L would be regulated by that of RdCVF2 (Leveillard and Ait-Ali, 2017).

RdCVF2 can also synergize with RdCVF2L by regulating transmembrane electrochemical gradients. The cellular Na/K-ATPase pump activity relies on ATP produced by glycolytic, rather than by ATP from mitochondrial respiratory chain (Dutka and Lamb, 2007). The Na/K-ATPase re-establishes the potassium and sodium gradients which are necessary to fire action potentials. Neurons, such as hippocampal pyramidal neurons expend a large fraction of the ATP they produce to maintain their required intracellular Na and K concentrations (Gerkau et al., 2019). RdCVF2 could increase locally, at the level of its receptor on hippocampal pyramidal neurons, the concentration of ATP produced from glucose by glycolysis. The addition of ATP generates functional LTP on hippocampal slices (Fujii, 2004). In addition, RdCVF2-mediated glycolysis can branch to the production of triglycerides that can participate in structural LTP, the reorganization of cytoskeletal architecture that produces new synaptic buttons (Dotti et al., 2014), similarly to RdCVF's ability to stimulate aerobic glycolysis to produce of triglycerides for cone outer segment renewal (Ait-Ali et al., 2015). A local action of RdCVF2 through the dendritic spines expressing its putative RdCVF2 cell-surface receptor and GLUT4 would explain the inability to restore the whole metabolism of the hippocampus (Ashrafi et al., 2017) (data not shown).

### The Implication of NXNL2 in Neurodegenerative Diseases

Oligomerization, phosphorylation at AT100 and AT8 epitopes and aggregation of TAU are hallmarks of tauopathies, such as AD. Neuropathologically AD is defined by the combined presence of extracellular amyloid-beta (Aβ) plaques and intracellular TAU NFT, but the MAPT gene encoding TAU was never found to be genetically associated with AD (Carmona et al., 2018). Similar to MAPT the NXNL2 gene is not genetically associated with AD (Lambert et al., 2009). This means that positive, but not negative genome wide association studies (GWAS) signals can lead to a conclusion on essential mechanisms of AD and leaves opened the possibility of NXNL2 participation in AD.

There is growing evidence for a close link between altered glucose metabolism and AD pathogenesis (Cho et al., 2021; Milstein and Ferris, 2021; Shippy and Ulland, 2020; Zhang et al., 2021). Aging, viewed as a slow steady accumulation of unrepaired oxidative damages, is the most relevant risk factor triggering AD as a disease-memory impairment of hippocampal function, the earliest affected brain region in AD. Redox enzymes are candidate regulators of the disease (Jia et al., 2021). Due to its dual function in regulating glucose uptake and redox status of TAU, the NXNL2 gene is positioned at a central place in this pathological aging scenario. Interestingly, another truncated thioredoxin, TRX80 prevents the accumulation of toxic amyloid β42 in the brain (Gil-Bea et al., 2012).

One of the most striking observations made on the Nxnl2^{-/-} mouse model is the parallel between memory dysfunction at 2-months that resembles mild-cognitive impairment predisposing to the development of AD (Karakaya et al., 2013; Knopman and Petersen, 2014), and the aggregation of TAU at 18-month, which is equivalent of NTF found in the brain of AD patients, after autopsy (Nelson et al., 2007). For the young Nxnl2^{-/-} mouse, LTP dysfunction is attributed to the lack of RdCVF2 and RdCVF2L that act synergistically. For the aged Nxnl2^{-/-} mouse, the protection against aggregation is believed to be the results of RdCVFL2 action, which can prevent TAU phosphorylation and its subsequent aggregation (Elachouri et al., 2015). However, since the treatment was administrated in young animals, TAU aggregation may be the result of metabolic and redox dysfunctions that occurred progressively throughout the life of mice.

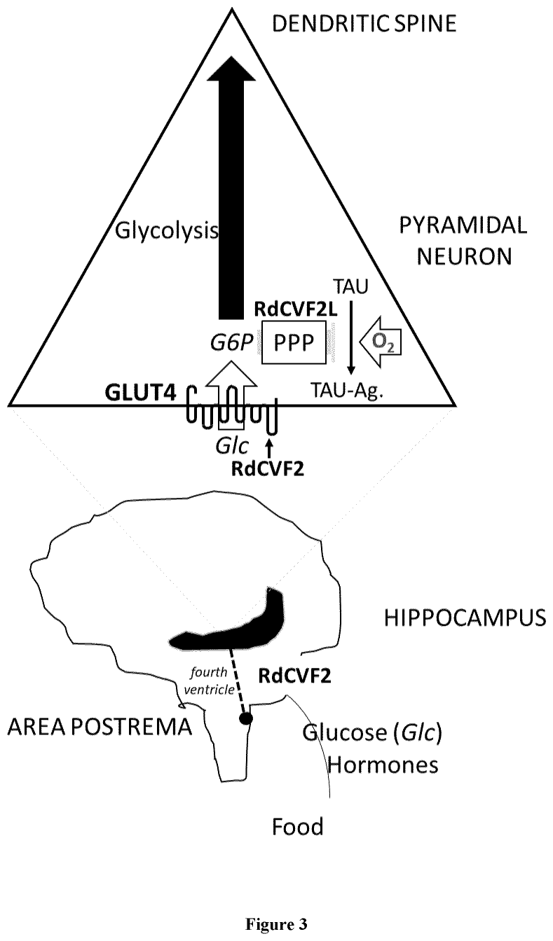

Altogether, these observations can be recapitulated in a working model (FIG. 3). In non-pathological conditions blood-borne satiety hormones and glucose, whose concentration increases after feeding, transfer directly to a subsets of neurons of the area postrema. Somehow, this signal triggers the production and the release of RdCVF2 in the cerebrospinal fluid, resulting in its circulation toward the pyramidal cells of the hippocampus. By binding to its receptor, still unidentified, at the surface of pyramidal neurons, RdCVF2 stimulates glucose uptake, which results in increased glycolysis used to sustain the extension of lipid membrane surface at the dendritic spines during memory acquisition or consolidation. In pathological conditions, such as those created by removing the Nxnl2 gene in the mouse, the shortage of glucose in pyramidal neurons reduces the repair activity of the two NADPH-dependent redox systems, and probably that of RdCVF2L. The metabolic plasticity of pyramidal neurons allows them to deal with this redox power dysfunction for some time, but aging adds to the burden, which results in a tauopathy. What is quite interesting is the parallel between the progression in two stages of the phenotype of the Nxnl2^{-/-} mouse and mild cognitive impairment that predispose later to dementia in human. The NXNL2 gene does not carry susceptibility alleles for AD so far, much as the MAPT gene encoding TAU, suggesting that while a genetic association in genome-wide association studies is informative, its absence cannot be a biological valid rejection criterion.

Since the products of the NXNL2 gene have, by analogy, the same therapeutic potential that those of the NXNL1 gene (Clerin et al., 2020), we delivered to newborn Nxnl2^{-/-} mice, RdCVF2 and RdCVF2L using an adeno-associated viral vector to correct the phenotype. The recombinant adeno-associated viral vector was injected in the heart and is then transferred to the brain in the young animals as they have not yet formed a functional blood-brain barrier. We found that both RdCVF2 and RdCVF2L partially prevents the LTP deficit, and interestingly, the combination of the two transgenes completely erases this deficit in a synergistic manner.

This demonstrates that the synergistic action of RdCVF2 on metabolism and RdCVF2L on redox power is coupled to the benefit of the function of hippocampal neurons. This system is similar to our findings in the retina between the actions of RdCVF and RdCVFL and we propose that glucose is the link between the two splice forms of the gene. The failure of all clinical trials carried out to date on AD with devastating consequences, leads the scientific community to look at new avenues.

Glucose, being the major energy source for neurons, the regulation of its metabolism is central in this reevaluation. The biological activity of the two products of the NXNL2 gene merits a special interest toward this goal.

Treating patients at the stage of mild-cognitive impairment with the products of the NXNL2 gene could be effective in preventing AD.

### REFERENCES

Throughout this application, various references describe the state of the art to which this invention pertains. The disclosures of these references are hereby incorporated by reference into the present disclosure.

Abraham, W.C., Jones, O.D., and Glanzman, D.L. (2019). Is plasticity of synapses the mechanism of long-term memory storage? NPJ Sci Learn 4, 9. 10.1038/s41539-019-0048-y.

Adlanmerini, M., Carpenter, B.J., Remsberg, J.R., Aubert, Y., Peed, L.C., Richter, H.J., and Lazar, M.A. (2019). Circadian lipid synthesis in brown fat maintains murine body temperature during chronic cold. Proceedings of the National Academy of Sciences of the United States of America 116, 18691-18699. 10.1073/pnas.1909883116.

Afridi, R., Lee, W.H., and Suk, K. (2020). Microglia Gone Awry: Linking Immunometabolism to Neurodegeneration. Frontiers in cellular neuroscience 14, 246. 10.3389/fncel.2020.00246.

Aincy, M., Meziane, H., Herault, Y., and Humeau, Y. (2018). Synaptic dysfunction in amygdala in intellectual disorder models. Progress in neuro-psychopharmacology & biological psychiatry 84, 392-397. 10.1016/j.pnpbp.2017.07.028.

Ait-Ali, N., Fridlich, R., Millet-Puel, G., Clerin, E., Delalande, F., Jaillard, C., Blond, F., Perrocheau, L., Reichman, S., Byrne, L.C., et al. (2015). Rod-derived cone viability factor promotes cone survival by stimulating aerobic glycolysis. Cell 161, 817-832. 10.1016/j.cell.2015.03.023.

Aizenman, E., Loring, R.H., Reynolds, I.J., and Rosenberg, P.A. (2020). The Redox Biology of Excitotoxic Processes: The NMDA Receptor, TOPA Quinone, and the Oxidative Liberation of Intracellular Zinc. Front Neurosci 14, 778. 10.3389/fnins.2020.00778.

Andersen, P., Morris, R., Amaral, D., Bliss, T., and O'Keefe, J. (2006). The hippocampus book (Oxford university press).

Aschauer, D.F., Kreuz, S., and Rumpel, S. (2013). Analysis of transduction efficiency, tropism and axonal transport of AAV serotypes 1, 2, 5, 6, 8 and 9 in the mouse brain. PLoS One 8, e76310. 10.1371/journal.pone.0076310.

Ashrafi, G., Wu, Z., Farrell, R.J., and Ryan, T.A. (2017). GLUT4 Mobilization Supports Energetic Demands of Active Synapses. Neuron 93, 606-615 e603. 10.1016/j.neuron.2016.12.020.

Ayhan, F., Kulkarni, A., Berto, S., Sivaprakasam, K., Douglas, C., Lega, B.C., and Konopka, G. (2021). Resolving cellular and molecular diversity along the hippocampal anterior-to-posterior axis in humans. Neuron. 10.1016/j.neuron.2021.05.003.

Bate, S.T., and Clark, R.A. (2014). The design and statistical analysis of animal experiments (Cambridge University Press).

Bolten, C.J., Kiefer, P., Letisse, F., Portais, J.C., and Wittmann, C. (2007). Sampling for metabolome analysis of microorganisms. Anal Chem 79, 3843-3849. 10.1021/ac0623888.

Braak, H., and Braak, E. (1991). Neuropathological stageing of Alzheimer-related changes. Acta Neuropathol 82, 239-259. 10.1007/BF00308809.

Bradbury, D.A., Simmons, T.D., Slater, K.J., and Crouch, S.P. (2000). Measurement of the ADP: ATP ratio in human leukaemic cell lines can be used as an indicator of cell viability, necrosis and apoptosis. J Immunol Methods 240, 79-92. 10.1016/s0022-1759(00 00178-2.

Buchner, D.A., Charrier, A., Srinivasan, E., Wang, L., Paulsen, M.T., Ljungman, M., Bridges, D., and Saltiel, A.R. (2015). Zinc finger protein 407 (ZFP407) regulates insulin-stimulated glucose uptake and glucose transporter 4 (Glut4) mRNA. J Biol Chem 290, 6376-6386. 10.1074/jbc.M114.623736.

Byrne, L.C., Dalkara, D., Luna, G., Fisher, S.K., Clerin, E., Sahel, J.A., Leveillard, T., and Flannery, J.G. (2015). Viral-mediated RdCVF and RdCVFL expression protects cone and rod photoreceptors in retinal degeneration. J Clin Invest 125, 105-116. 10.1172/JCI65654.

Camacho, E.T., Brager, D., Elachouri, G., Korneyeva, T., Millet-Puel, G., Sahel, J.A., and Leveillard, T. (2019). A Mathematical Analysis of Aerobic Glycolysis Triggered by Glucose Uptake in Cones. Sci Rep 9, 4162. 10.1038/s41598-019-39901-z.

Carmona, S., Hardy, J., and Guerreiro, R. (2018). The genetic landscape of Alzheimer disease. Handb Clin Neurol 148, 395-408. 10.1016/B978-0-444-64076-5.00026-0.

Castagne, V., Porsolt, R.D., and Moser, P. (2009). Use of latency to immobility improves detection of antidepressant-like activity in the behavioral despair test in the mouse. European journal of pharmacology 616, 128-133. 10.1016/j.ejphar.2009.06.018.

Chalmel, F., Leveillard, T., Jaillard, C., Lardenois, A., Berdugo, N., Morel, E., Koehl, P., Lambrou, G., Holmgren, A., Sahel, J.A., and Poch, O. (2007). Rod-derived Cone Viability Factor-2 is a novel bifunctional-thioredoxin-like protein with therapeutic potential. BMC Mol Biol 8, 74. 10.1186/1471-2199-8-74.

Chandel, N.S. (2015). Navigating metabolism.

Chinchore, Y., Begaj, T., Wu, D., Drokhlyansky, E., and Cepko, C.L. (2017). Glycolytic reliance promotes anabolism in photoreceptors. eLife 6. 10.7554/eLife.25946.

Cho, S., Lee, H., and Seo, J. (2021). Impact of Genetic Risk Factors for Alzheimer's Disease on Brain Glucose Metabolism. Mol Neurobiol 58, 2608-2619. 10.1007/s12035-021-02297-x. Clayton, J.A., and Collins, F.S. (2014). Policy: NIH to balance sex in cell and animal studies. Nature 509, 282-283. 10.1038/509282a.

Clerin, E., Marussig, M., Sahel, J.A., and Leveillard, T. (2020). Metabolic and Redox Signaling of the Nucleoredoxin-Like-1 Gene for the Treatment of Genetic Retinal Diseases. Int J Mol Sci 21. 10.3390/ijms21051625.

Crawley, J.N. (2007). What's wrong with my mouse?: behavioral phenotyping of transgenic and knockout mice (John Wiley & Sons).

Cronin, T., Raffelsberger, W., Lee-Rivera, I., Jaillard, C., Niepon, M.L., Kinzel, B., Clerin, E., Petrosian, A., Picaud, S., Poch, O., et al. (2010). The disruption of the rod-derived cone viability gene leads to photoreceptor dysfunction and susceptibility to oxidative stress. Cell Death Differ 17, 1199-1210. 10.1038/cdd.2010.2.

da Conceicao, E.P.S., Morrison, S.F., Cano, G., Chiavetta, P., and Tupone, D. (2020). Median preoptic area neurons are required for the cooling and febrile activations of brown adipose tissue thermogenesis in rat. Sci Rep 10, 18072. 10.1038/s41598-020-74272-w.

Dalkara, D., Byrne, L.C., Lee, T., Hoffmann, N.V., Schaffer, D.V., and Flannery, J.G. (2012). Enhanced gene delivery to the neonatal retina through systemic administration of tyrosine-mutated AAV9. Gene Ther 19, 176-181. 10.1038/gt.2011.163.

Damkier, H.H., Brown, P.D., and Praetorius, J. (2013). Cerebrospinal fluid secretion by the choroid plexus. Physiol Rev 93, 1847-1892. 10.1152/physrev.00004.2013.

Dawson, H.N., Ferreira, A., Eyster, M.V., Ghoshal, N., Binder, L.I., and Vitek, M.P. (2001). Inhibition of neuronal maturation in primary hippocampal neurons from tau deficient mice. J Cell Sci 114, 1179-1187.

Dos Santos, J.P.A., Vizuete, A., Hansen, F., Biasibetti, R., and Goncalves, C.A. (2018). Early and Persistent O-GlcNAc Protein Modification in the Streptozotocin Model of Alzheimer's Disease. J Alzheimers Dis 61, 237-249. 10.3233/JAD-170211.

Dotti, C.G., Esteban, J.A., and Ledesma, M.D. (2014). Lipid dynamics at dendritic spines. Front Neuroanat 8, 76. 10.3389/fnana.2014.00076.

Dubos, A., Meziane, H., Iacono, G., Curie, A., Riet, F., Martin, C., Loaec, N., Birling, M.C., Selloum, M., Normand, E., et al. (2018). A new mouse model of ARX dup24 recapitulates the patients' behavioral and fine motor alterations. Hum Mol Genet 27, 2138-2153. 10.1093/hmg/ddyl22.

Duchon, A., Del Mar Muniz Moreno, M., Martin Lorenzo, S., Silva de Souza, M.P., Chevalier, C., Nalesso, V., Meziane, H., Loureiro de Sousa, P., Noblet, V., Armspach, J.P., et al. (2021). Multi-influential genetic interactions alter behaviour and cognition through six main biological cascades in Down syndrome mouse models. Hum Mol Genet 30, 771-788. 10.1093/hmg/ddab012.

Duran, J., Saez, I., Gruart, A., Guinovart, J.J., and Delgado-Garcia, J.M. (2013). Impairment in long-term memory formation and learning-dependent synaptic plasticity in mice lacking glycogen synthase in the brain. J Cereb Blood Flow Metab 33, 550-556. 10.1038/jcbfm.2012.200.

Dutka, T.L., and Lamb, G.D. (2007). Na+-K+ pumps in the transverse tubular system of skeletal muscle fibers preferentially use ATP from glycolysis. Am J Physiol Cell Physiol 293, C967-977. 10.1152/ajpcell.00132.2007.

Ebersole, J., Rose, G., Eid, T., Behar, K., and Patrylo, P. (2021). Altered hippocampal astroglial metabolism is associated with aging and preserved spatial learning and memory. Neurobiol Aging 102, 188-199. 10.1016/j.neurobiolaging.2021.02.015.

Elachouri, G., Lee-Rivera, I., Clerin, E., Argentini, M., Fridlich, R., Blond, F., Ferracane, V., Yang, Y., Raffelsberger, W., Wan, J., et al. (2015). Thioredoxin rod-derived cone viability factor protects against photooxidative retinal damage. Free Radic Biol Med 81, 22-29. 10.1016/j.freeradbiomed.2015.01.003.

Ellacott, K.L., Morton, G.J., Woods, S.C., Tso, P., and Schwartz, M.W. (2010). Assessment of feeding behavior in laboratory mice. Cell Metab 12, 10-17. 10.1016/j.cmet.2010.06.001.

Fischer, A.W., Cannon, B., and Nedergaard, J. (2018). Optimal housing temperatures for mice to mimic the thermal environment of humans: An experimental study. Molecular metabolism 7, 161-170. 10.1016/j.molmet.2017.10.009.

Fridlich, R., Delalande, F., Jaillard, C., Lu, J., Poidevin, L., Cronin, T., Perrocheau, L., Millet-Puel, G., Niepon, M.L., Poch, O., et al. (2009). The thioredoxin-like protein rod-derived cone viability factor (RdCVFL) interacts with TAU and inhibits its phosphorylation in the retina. Molecular & cellular proteomics: MCP 8, 1206-1218. 10.1074/mcp.M800406-MCP200.

Fry, M., Hoyda, T.D., and Ferguson, A.V. (2007). Making sense of it: roles of the sensory circumventricular organs in feeding and regulation of energy homeostasis. Exp Biol Med (Maywood) 232, 14-26.

Fujii, S. (2004). ATP- and adenosine-mediated signaling in the central nervous system: the role of extracellular ATP in hippocampal long-term potentiation. J Pharmacol Sci 94, 103-106. 10.1254/jphs.94.103.

Fultz, N.E., Bonmassar, G., Setsompop, K., Stickgold, R.A., Rosen, B.R., Polimeni, J.R., and Lewis, L.D. (2019). Coupled electrophysiological, hemodynamic, and cerebrospinal fluid oscillations in human sleep. Science 366, 628-631. 10.1126/science.aax5440.

Gaspar, R.C., Pauli, J.R., Shulman, G.I., and Munoz, V.R. (2021). An update on brown adipose tissue biology: a discussion of recent findings. American journal of physiology. Endocrinology and metabolism 320, E488-E495. 10.1152/ajpendo.00310.2020.

Gerkau, N.J., Lerchundi, R., Nelson, J.S.E., Lantermann, M., Meyer, J., Hirrlinger, J., and Rose, C.R. (2019). Relation between activity-induced intracellular sodium transients and ATP dynamics in mouse hippocampal neurons. J Physiol 597, 5687-5705. 10.1113/JP278658.

Gil-Bea, F., Akterin, S., Persson, T., Mateos, L., Sandebring, A., Avila-Carino, J., Gutierrez-Rodriguez, A., Sundstrom, E., Holmgren, A., Winblad, B., and Cedazo-Minguez, A. (2012). Thioredoxin-80 is a product of alpha-secretase cleavage that inhibits amyloid-beta aggregation and is decreased in Alzheimer's disease brain. EMBO Mol Med 4, 1097-1111. 10.1002/emmm.201201462.

Gomez-Nieto, R., Hormigo, S., and Lopez, D.E. (2020). Prepulse Inhibition of the Auditory Startle Reflex Assessment as a Hallmark of Brainstem Sensorimotor Gating Mechanisms. Brain Sci 10. 10.3390/brainsci10090639.

Gordon, C.J. (2017). The mouse thermoregulatory system: Its impact on translating biomedical data to humans. Physiology & behavior 179, 55-66. 10.1016/j.physbeh.2017.05.026.

Greene, A.E., Todorova, M.T., and Seyfried, T.N. (2003). Perspectives on the metabolic management of epilepsy through dietary reduction of glucose and elevation of ketone bodies. J Neurochem 86, 529-537. 10.1046/j.1471-4159.2003.01862.x.

Grieger, J.C., Soltys, S.M., and Samulski, R.J. (2016). Production of Recombinant Adeno-associated Virus Vectors Using Suspension HEK293 Cells and Continuous Harvest of Vector From the Culture Media for GMP FIX and FLT1 Clinical Vector. Mol Ther 24, 287-297. 10.1038/mt.2015.187.

Gyparaki, M.T., Arab, A., Sorokina, E.M., Santiago-Ruiz, A.N., Bohrer, C.H., Xiao, J., and Lakadamyali, M. (2021). Tau forms oligomeric complexes on microtubules that are distinct from tau aggregates. Proc Natl Acad Sci U S A 118. 10.1073/pnas.2021461118.

Habib, N., Li, Y., Heidenreich, M., Swiech, L., Avraham-Davidi, I., Trombetta, J.J., Hession, C., Zhang, F., and Regev, A. (2016). Div-Seq: Single-nucleus RNA-Seq reveals dynamics of rare adult newborn neurons. Science 353, 925-928. 10.1126/science.aad7038.

Haunhorst, P., Berndt, C., Eitner, S., Godoy, J.R., and Lillig, C.H. (2010). Characterization of the human monothiol glutaredoxin 3 (PICOT) as iron-sulfur protein. Biochem Biophys Res Commun 394, 372-376. 10.1016/j.bbrc.2010.03.016.

Herrero, E., and de la Torre-Ruiz, M.A. (2007). Monothiol glutaredoxins: a common domain for multiple functions. Cellular and molecular life sciences: CMLS 64, 1518-1530. 10.1007/s00018-007-6554-8.

Hu, Q., Noll, R.J., Li, H., Makarov, A., Hardman, M., and Graham Cooks, R. (2005). The Orbitrap: a new mass spectrometer. J Mass Spectrom 40, 430-443. 10.1002/jms.856.

Huang, T.T., Leu, D., and Zou, Y. (2015). Oxidative stress and redox regulation on hippocampal-dependent cognitive functions. Arch Biochem Biophys 576, 2-7. 10.1016/j.abb.2015.03.014.

Hur, J., Smith, J.F., DeYoung, K.A., Anderson, A.S., Kuang, J., Kim, H.C., Tillman, R.M., Kuhn, M., Fox, A.S., and Shackman, A.J. (2020). Anxiety and the Neurobiology of Temporally Uncertain Threat Anticipation. The Journal of neuroscience: the official journal of the Society for Neuroscience 40, 7949-7964. 10.1523/JNEUROSCI.0704-20.2020.

Jaillard, C., Mouret, A., Niepon, M.L., Clerin, E., Yang, Y., Lee-Rivera, I., Ait-Ali, N., Millet-Puel, G., Cronin, T., Sedmak, T., et al. (2012). Nxnl2 splicing results in dual functions in neuronal cell survival and maintenance of cell integrity. Hum Mol Genet 21, 2298-2311. 10.1093/hmg/dds050.

Jia, J., Zeng, X., Xu, G., and Wang, Z. (2021). The Potential Roles of Redox Enzymes in Alzheimer's Disease: Focus on Thioredoxin. ASN Neuro 13, 1759091421994351. 10.1177/1759091421994351.

Jimenez, J.C., Su, K., Goldberg, A.R., Luna, V.M., Biane, J.S., Ordek, G., Zhou, P., Ong, S.K., Wright, M.A., Zweifel, L., et al. (2018). Anxiety Cells in a Hippocampal-Hypothalamic Circuit. Neuron 97, 670-683 e676. 10.1016/j.neuron.2018.01.016.

Karakaya, T., Fusser, F., Schroder, J., and Pantel, J. (2013). Pharmacological Treatment of Mild Cognitive Impairment as a Prodromal Syndrome of Alzheimer's Disease. Curr Neuropharmacol 11, 102-108. 10.2174/157015913804999487.

Kataoka, N., Hioki, H., Kaneko, T., and Nakamura, K. (2014). Psychological stress activates a dorsomedial hypothalamus-medullary raphe circuit driving brown adipose tissue thermogenesis and hyperthermia. Cell metabolism 20, 346-358. 10.1016/j.cmet.2014.05.018.

Keller, D., Ero, C., and Markram, H. (2018). Cell Densities in the Mouse Brain: A Systematic Review. Front Neuroanat 12, 83. 10.3389/fnana.2018.00083.

Kerchner, G.A., and Nicoll, R.A. (2008). Silent synapses and the emergence of a postsynaptic mechanism for LTP. Nat Rev Neurosci 9, 813-825. 10.1038/nrn2501.

Kiecker, C. (2018). The origins of the circumventricular organs. J Anat 232, 540-553. 10.1111/joa.12771.

Kiefer, P., Nicolas, C., Letisse, F., and Portais, J.C. (2007). Determination of carbon labeling distribution of intracellular metabolites from single fragment ions by ion chromatography tandem mass spectrometry. Anal Biochem 360, 182-188. 10.1016/j.ab.2006.06.032.

Kim, J.H., Lee, S.R., Li, L.H., Park, H.J., Park, J.H., Lee, K.Y., Kim, M.K., Shin, B.A., and Choi, S.Y. (2011). High cleavage efficiency of a 2A peptide derived from porcine teschovirus-1 in human cell lines, zebrafish and mice. PLoS One 6, e18556. 10.1371/journal.pone.0018556.

Knopman, D.S., and Petersen, R.C. (2014). Mild cognitive impairment and mild dementia: a clinical perspective. Mayo Clin Proc 89, 1452-1459. 10.1016/j.mayocp.2014.06.019.

Koepsell, H. (2020). Glucose transporters in brain in health and disease. Pflugers Arch 472, 1299-1343. 10.1007/s00424-020-02441-x.

La-Vu, M., Tobias, B.C., Schuette, P.J., and Adhikari, A. (2020). To Approach or Avoid: An Introductory Overview of the Study of Anxiety Using Rodent Assays. Frontiers in behavioral neuroscience 14, 145. 10.3389/fnbeh.2020.00145.

Lambard, S., Reichman, S., Berlinicke, C., Niepon, M.L., Goureau, O., Sahel, J.A., Leveillard, T., and Zack, D.J. (2010). Expression of rod-derived cone viability factor: dual role of CRX in regulating promoter activity and cell-type specificity. PLoS One 5, e13075. 10.1371/journal.pone.0013075.

Lambert, J.C., Heath, S., Even, G., Campion, D., Sleegers, K., Hiltunen, M., Combarros, O., Zelenika, D., Bullido, M.J., Tavernier, B., et al. (2009). Genome-wide association study identifies variants at CLU and CR1 associated with Alzheimer's disease. Nat Genet 41, 1094-1099. 10.1038/ng.439.

Lamprianou, S., Vacaresse, N., Suzuki, Y., Meziane, H., Buxbaum, J.D., Schlessinger, J., and Harroch, S. (2006). Receptor protein tyrosine phosphatase gamma is a marker for pyramidal cells and sensory neurons in the nervous system and is not necessary for normal development. Mol Cell Biol 26, 5106-5119. 10.1128/MCB.00101-06.

Le Roux, N., Cabezas, C., Bohm, U.L., and Poncer, J.C. (2013). Input-specific learning rules at excitatory synapses onto hippocampal parvalbumin-expressing interneurons. J Physiol 591, 1809-1822. 10.1113/jphysiol.2012.245852.

Leveillard, T. (2015). Cancer metabolism of cone photoreceptors. Oncotarget 6, 32285-32286. 10.18632/oncotarget.5963.

Leveillard, T., and Ait-Ali, N. (2017). Cell Signaling with Extracellular Thioredoxin and Thioredoxin-Like Proteins: Insight into Their Mechanisms of Action. Oxid Med Cell Longev 2017, 8475125. 10.1155/2017/8475125.

Léveillard, T., Ouechtati, F., Corsi, M., and Blond, F. (2021). Expression profile of the nucleoredoxin-like 2 gene in the mouse brain using a beta-galactosidase knock-in reporter strain. EBRAINS, 10.25493/4975-NDG.

Leveillard, T., and Sahel, J.A. (2017). Metabolic and redox signaling in the retina. Cell Mol Life Sci 74, 3649-3665. 10.1007/s00018-016-2318-7.

Li, F., and Tsien, J.Z. (2009). Memory and the NMDA receptors. N Engl J Med 361, 302-303. 10.1056/NEJMcibr0902052.

Lian, X.Y., Khan, F.A., and Stringer, J.L. (2007). Fructose-1,6-bisphosphate has anticonvulsant activity in models of acute seizures in adult rats. J Neurosci 27, 12007-12011. 10.1523/JNEUROSCI.3163-07.2007.

Lipton, S.A., Choi, Y.B., Takahashi, H., Zhang, D., Li, W., Godzik, A., and Bankston, L.A. (2002). Cysteine regulation of protein function—as exemplified by NMDA-receptor modulation. Trends Neurosci 25, 474-480. 10.1016/s0166-2236(02)02245-2.

Love, D.C., and Hanover, J.A. (2005). The hexosamine signaling pathway: deciphering the “O-GlcNAc code”. Sci STKE 2005, re13. 10.1126/stke.3122005re13.

MacDonald, A.J., Yang, Y.H.C., Cruz, A.M., Beall, C., and Ellacott, K.L.J. (2021). Brain-Body Control of Glucose Homeostasis-Insights From Model Organisms. Front Endocrinol (Lausanne) 12, 662769. 10.3389/fendo.2021.662769.

Maeda, S., Sahara, N., Saito, Y., Murayama, S., Ikai, A., and Takashima, A. (2006). Increased levels of granular tau oligomers: an early sign of brain aging and Alzheimer's disease. Neurosci Res 54, 197-201. 10.1016/j.neures.2005.11.009.

Malia, T.J., Teplyakov, A., Ernst, R., Wu, S.J., Lacy, E.R., Liu, X., Vandermeeren, M., Mercken, M., Luo, J., Sweet, R.W., and Gilliland, G.L. (2016). Epitope mapping and structural basis for the recognition of phosphorylated tau by the anti-tau antibody AT8. Proteins 84, 427-434. 10.1002/prot.24988.

Maolood, N., and Meister, B. (2009). Protein components of the blood-brain barrier (BBB) in the brainstem area postrema-nucleus tractus solitarius region. J Chem Neuroanat 37, 182-195. 10.1016/j.jchemneu.2008.12.007.

Mckinley, M.J., Denton, D.A., Ryan, P.J., Yao, S.T., Stefanidis, A., and Oldfield, B.J. (2019). From sensory circumventricular organs to cerebral cortex: Neural pathways controlling thirst and hunger. J Neuroendocrinol 31, e12689. 10.1111/jne.12689.

McNay, E.C., and Pearson-Leary, J. (2020). GluT4: A central player in hippocampal memory and brain insulin resistance. Exp Neurol 323, 113076. 10.1016/j.expneurol.2019.113076.

Medlej, Y., Salah, H., Wadi, L., Saad, S., Asdikian, R., Karnib, N., Ghazal, D., Bashir, B., Allam, J., and Obeid, M. (2019). Overview on Emotional Behavioral Testing in Rodent Models of Pediatric Epilepsy. Methods Mol Biol 2011, 345-367. 10.1007/978-1-4939-9554-7_20.

Mei, X., Chaffiol, A., Kole, C., Yang, Y., Millet-Puel, G., Clerin, E., Ait-Ali, N., Bennett, J., Dalkara, D., Sahel, J.A., et al. (2016). The Thioredoxin Encoded by the Rod-Derived Cone Viability Factor Gene Protects Cone Photoreceptors Against Oxidative Stress. Antioxid Redox Signal 24, 909-923. 10.1089/ars.2015.6509.

Miller, C.G., Holmgren, A., Arner, E.S.J., and Schmidt, E.E. (2018). NADPH-dependent and -independent disulfide reductase systems. Free Radic Biol Med 127, 248-261. 10.1016/j.freeradbiomed.2018.03.051.

Milstein, J.L., and Ferris, H.A. (2021). The brain as an insulin-sensitive metabolic organ. Mol Metab, 101234. 10.1016/j.molmet.2021.101234.

Nanavaty, N., Lin, L., Hinckley, S.H., and Kuret, J. (2017). Detection and Quantification Methods for Fibrillar Products of In Vitro Tau Aggregation Assays. Methods Mol Biol 1523, 101-111. 10.1007/978-1-4939-6598-4_6.

Naso, M.F., Tomkowicz, B., Perry, W.L., 3rd, and Strohl, W.R. (2017). Adeno-Associated Virus (AAV) as a Vector for Gene Therapy. BioDrugs 31, 317-334. 10.1007/s40259-017-0234-5.

Nelson, P.T., Jicha, G.A., Schmitt, F.A., Liu, H., Davis, D.G., Mendiondo, M.S., Abner, E.L., and Markesbery, W.R. (2007). Clinicopathologic correlations in a large Alzheimer disease center autopsy cohort: neuritic plaques and neurofibrillary tangles “do count” when staging disease severity. J Neuropathol Exp Neurol 66, 1136-1146. 10.1097/nen.0b013e31815c5efb.

Price, C.J., Hoyda, T.D., and Ferguson, A.V. (2008). The area postrema: a brain monitor and integrator of systemic autonomic state. Neuroscientist 14, 182-194. 10.1177/1073858407311100.

Rath, A., Glibowicka, M., Nadeau, V.G., Chen, G., and Deber, C.M. (2009). Detergent binding explains anomalous SDS-PAGE migration of membrane proteins. Proc Natl Acad Sci U S A 106, 1760-1765. 10.1073/pnas.0813167106.

Ren, X., Zou, L., Zhang, X., Branco, V., Wang, J., Carvalho, C., Holmgren, A., and Lu, J. (2017). Redox Signaling Mediated by Thioredoxin and Glutathione Systems in the Central Nervous System. Antioxid Redox Signal 27, 989-1010. 10.1089/ars.2016.6925.

Rodrigues, K., Pereira, R.M., de Campos, T.D.P., de Moura, R.F., da Silva, A.S.R., Cintra, D.E., Ropelle, E.R., Pauli, J.R., de Araujo, M.B., and de Moura, L.P. (2018). The Role of Physical Exercise to Improve the Browning of White Adipose Tissue via POMC Neurons. Frontiers in cellular neuroscience 12, 88. 10.3389/fncel.2018.00088.

Sakayori, N., Kato, S., Sugawara, M., Setogawa, S., Fukushima, H., Ishikawa, R., Kida, S., and Kobayashi, K. (2019). Motor skills mediated through cerebellothalamic tracts projecting to the central lateral nucleus. Molecular brain 12, 13. 10.1186/s13041-019-0431-x.

Satishchandra, P., and Mathew, T. (2008). Handbook of clinical neurology, vol 87 (3rd series) malformations of the nervous system. BMJ Publishing Group Ltd.

Schwantje, M., Verhagen, L.M., van Hasselt, P.M., and Fuchs, S.A. (2020). Glucose transporter type 1 deficiency syndrome and the ketogenic diet. J Inherit Metab Dis 43, 216-222. 10.1002/jimd.12175.

Shippy, D.C., and Ulland, T.K. (2020). Microglial Immunometabolism in Alzheimer's Disease. Front Cell Neurosci 14, 563446. 10.3389/fncel.2020.563446.

Soeda, Y., Yoshikawa, M., Almeida, O.F., Sumioka, A., Maeda, S., Osada, H., Kondoh, Y., Saito, A., Miyasaka, T., Kimura, T., et al. (2015). Toxic tau oligomer formation blocked by capping of cysteine residues with 1,2-dihydroxybenzene groups. Nat Commun 6, 10216. 10.1038/ncomms10216.

Stukalin, Y., Lan, A., and Einat, H. (2020). Revisiting the validity of the mouse tail suspension test: Systematic review and meta-analysis of the effects of prototypic antidepressants. Neuroscience and biobehavioral reviews 112, 39-47. 10.1016/j.neubiorev.2020.01.034.

Tokar, D., van Ekeris, L., Linser, P.J., and Ochrietor, J.D. (2017). Characterization of the Expression of Basigin Gene Products Within the Pineal Gland of Mice. Cell Mol Neurobiol 37, 1141-1145. 10.1007/s10571-016-0441-5.

Uchihara, T. (2020). Neurofibrillary changes undergoing morphological and biochemical changes—How does tau with the profile shift of from four repeat to three repeat spread in Alzheimer brain? Neuropathology 40, 450-459. 10.1111/neup.12669.

Ullah, F., Liang, H., Niedermayer, G., Munch, G., and Gyengesi, E. (2020). Evaluation of Phytosomal Curcumin as an Anti-inflammatory Agent for Chronic Glial Activation in the GFAP-IL6 Mouse Model. Front Neurosci 14, 170. 10.3389/fnins.2020.00170.

Valenzuela, D.M., Murphy, A.J., Frendewey, D., Gale, N.W., Economides, A.N., Auerbach, W., Poueymirou, W.T., Adams, N.C., Rojas, J., Yasenchak, J., et al. (2003). High-throughput engineering of the mouse genome coupled with high-resolution expression analysis. Nat Biotechnol 21, 652-659. 10.1038/nbt822.

Vasireddy, V., Mills, J.A., Gaddameedi, R., Basner-Tschakarjan, E., Kohnke, M., Black, A.D., Alexandrov, K., Zhou, S., Maguire, A.M., Chung, D.C., et al. (2013). AAV-mediated gene therapy for choroideremia: preclinical studies in personalized models. PLoS One 8, e61396. 10.1371/journal.pone.0061396.

Wang, Q.P., Guan, J.L., Pan, W., Kastin, A.J., and Shioda, S. (2008). A diffusion barrier between the area postrema and nucleus tractus solitarius. Neurochem Res 33, 2035-2043. 10.1007/s11064-008-9676-y.

Webster, S.J., Bachstetter, A.D., Nelson, P.T., Schmitt, F.A., and Van Eldik, L.J. (2014). Using mice to model Alzheimer's dementia: an overview of the clinical disease and the preclinical behavioral changes in 10 mouse models. Frontiers in genetics 5, 88. 10.3389/fgene.2014.00088.

Wesseling, H., Mair, W., Kumar, M., Schlaffner, C.N., Tang, S., Beerepoot, P., Fatou, B., Guise, A.J., Cheng, L., Takeda, S., et al. (2020). Tau PTM Profiles Identify Patient Heterogeneity and Stages of Alzheimer's Disease. Cell 183, 1699-1713 e1613. 10.1016/j.cell.2020.10.029.

Wilson, S.G., and Mogil, J.S. (2001). Measuring pain in the (knockout) mouse: big challenges in a small mammal. Behav Brain Res 125, 65-73. 10.1016/s0166-4328(01)00281-9.

Wolloscheck, T., Kunst, S., Kelleher, D.K., and Spessert, R. (2015). Transcriptional regulation of nucleoredoxin-like genes takes place on a daily basis in the retina and pineal gland of rats. Visual neuroscience 32, E002. 10.1017/S0952523814000352.

Wu, L., Mashego, M.R., van Dam, J.C., Proell, A.M., Vinke, J.L., Ras, C., van Winden, W.A., van Gulik, W.M., and Heijnen, J.J. (2005). Quantitative analysis of the microbial metabolome by isotope dilution mass spectrometry using uniformly 13C-labeled cell extracts as internal standards. Anal Biochem 336, 164-171. 10.1016/j.ab.2004.09.001.

Xiong, Y., Uys, J.D., Tew, K.D., and Townsend, D.M. (2011). S-glutathionylation: from molecular mechanisms to health outcomes. Antioxidants & redox signaling 15, 233-270. 10.1089/ars.2010.3540.

Yang, Y.R., Song, S., Hwang, H., Jung, J.H., Kim, S.J., Yoon, S., Hur, J.H., Park, J.I., Lee, C., Nam, D., et al. (2017). Memory and synaptic plasticity are impaired by dysregulated hippocampal O-GlcNAcylation. Sci Rep 7, 44921. 10.1038/srep44921.

Yoshida, H., and Goedert, M. (2006). Sequential phosphorylation of tau protein by cAMP-dependent protein kinase and SAPK4/p38delta or JNK2 in the presence of heparin generates the AT100 epitope. J Neurochem 99, 154-164. 10.1111/j.1471-4159.2006.04052.x.

Yu, D., Corbett, B., Yan, Y., Zhang, G.X., Reinhart, P., Cho, S.J., and Chin, J. (2012). Early cerebrovascular inflammation in a transgenic mouse model of Alzheimer's disease. Neurobiol Aging 33, 2942-2947. 10.1016/j.neurobiolaging.2012.02.023.

Zhang, J., Ye, Z.W., Singh, S., Townsend, D.M., and Tew, K.D. (2018). An evolving understanding of the S-glutathionylation cycle in pathways of redox regulation. Free Radic Biol Med 120, 204-216. 10.1016/j.freeradbiomed.2018.03.038.

Zhang, S., Jin, X., You, Z., Wang, S., Lim, G., Yang, J., McCabe, M., Li, N., Marota, J., Chen, L., and Mao, J. (2014). Persistent nociception induces anxiety-like behavior in rodents: role of endogenous neuropeptide S. Pain 155, 1504-1515. 10.1016/j.pain.2014.04.026.

Zhang, T., Xie, P., Dong, Y., Liu, Z., Zhou, F., Pan, D., Huang, Z., Zhai, Q., Gu, Y., Wu, Q., et al. (2020). High-throughput discovery of genetic determinants of circadian misalignment. PLoS Genet 16, e1008577. 10.1371/journal.pgen.1008577.

Zhang, X., Alshakhshir, N., and Zhao, L. (2021). Glycolytic Metabolism, Brain Resilience, and Alzheimer's Disease. Front Neurosci 15, 662242. 10.3389/fnins.2021.662242.

1. A method of treating a tauopathy in a patient in need thereof comprising administering to the patient a therapeutically effective amount of a first polynucleotide encoding for the short isoform of the NXNL2 gene, Rod-derived Cone Viability Factor (RdCVF2) and of a second polynucleotide encoding for the long isoform of the NXNL2 gene, RdCVF2L.

2. The method of claim 1 wherein the patient is at a stage of mild-cognitive impairment.

3. The method of claim 1 wherein the first polynucleotide and the second polynucleotide are contained in separate expression vectors.

4. The method of claim 1 wherein the first polynucleotide and the second polynucleotide are contained in a single vector.

5. The method of claim 4 wherein the single vector is a viral vector.

6. The method of claim 5 wherein the viral vector is an AVV vector.

7. The method of claim 3 wherein the separate expression vectors are viral vectors.

8. The method of claim 7 wherein the viral vectors are AVV vectors.


SEQUENCE LISTING (2 sequences)

SEQ ID NO: 1 - protein, 101 aa, Homo sapiens
MVDILGERHLVTCKGATVEAEAALQNKVVALYFAAARCAPSRDFTPLLCDFYTALVAEARRPAPFEVVFVSADGSSQEMLDFMRELHGAWLALPFHDPYRH

SEQ ID NO: 2 - protein, 156 aa, Homo sapiens
MVDILGERHLVTCKGATVEAEAALQNKVVALYFAAARCAPSRDFTPLLCDFYTALVAEARRPAPFEVVFVSADGSSQEMLDFMRELHGAWLALPFHDPYRHELRKRYNVTAIPKLVIVKQNGEVITNKGRKQIRERGLACFQDWVEAADIFQNFSV